SEQUENCE LISTING (9 sequences)

SEQ ID NO: 1 - protein, 22 aa, ARTIFICIAL SEQUENCE
  POLY(GLU)-POLY(GLY)-RGD PEPTIDE FOR ELECTROSTATIC COATING
EEEEEEEEEEEEGGGGGGGRGD

SEQ ID NO: 2 - protein, 23 aa, ARTIFICIAL SEQUENCE
  POLY(GLU)-POLY(GLY)-RGDS PEPTIDE FOR ELECTROSTATIC COATING
EEEEEEEEEEEEGGGGGGGRGDS

SEQ ID NO: 3 - protein, 23 aa, ARTIFICIAL SEQUENCE
  POLY(GLU)-POLY(GLY)-RDGS PEPTIDE FOR ELECTROSTATIC COATING
EEEEEEEEEEEEGGGGGGGRDGS

SEQ ID NO: 4 - protein, 9 aa, ARTIFICIAL SEQUENCE
  FRAGMENT OF PEPTIDE FOR ELECTROSTATIC COATING
CDCRGDCFC

SEQ ID NO: 5 - protein, 9 aa, ARTIFICIAL SEQUENCE
  FRAGMENT OF PEPTIDE FOR ELECTROSTATIC COATING
CAQSNNKDC

SEQ ID NO: 6 - protein, 9 aa, ARTIFICIAL SEQUENCE
  FRAGMENT OF PEPTIDE FOR ELECTROSTATIC COATING
CGNKRTRGC

SEQ ID NO: 7 - protein, 9 aa, ARTIFICIAL SEQUENCE
  FRAGMENT OF PEPTIDE FOR ELECTROSTATIC COATING
CARSKNKDC

SEQ ID NO: 8 - protein, 10 aa, ARTIFICIAL SEQUENCE
  FRAGMENT OF PEPTIDE FOR ELECTROSTATIC COATING
PQRRSARLSA

SEQ ID NO: 9 - protein, 34 aa, ARTIFICIAL SEQUENCE
  FRAGMENT OF PEPTIDE FOR ELECTROSTATIC COATING
AKVKDEPQRRSARLSAKPAPPKPEPKPKKAPAKK

(19) United States
(12) Patent Application Publication
(10) Pub. No.: US 2010/0196492 A1
(43) Pub. Date: Aug. 5, 2010

(54) ELECTROSTATIC COATING OF PARTICLES FOR DRUG DELIVERY

(76) Inventors: Jordan J. Green, Arlington, MA (US); Daniel G. Anderson, Sudbury, MA (US); Robert S. Langer, Newton, MA (US); Eugene Chiu, Auberndale, MA (US); Elizaveta S. Leshchiner, Watertown, MA (US)

Correspondence Address: WOLF GREENFIELD & SACKS, P.C., 600 ATLANTIC AVENUE, BOSTON, MA, 02210-2206, US

(21) Appl. No.: 12/530,330

(22) PCT Filed: Mar. 7, 2008
(86) PCT No.: PCT/US08/56168

§ 371 Date: Mar. 19, 2010

Related U.S. Application Data
(60) Provisional application No. 60/893,703, filed on Mar. 8, 2007.

(51) Int. Cl. A61K 9/32; A61K 38/02; A61K 38/16; A61K 31/70; A61K 31/7088; A61P 29/00; A61P 35/00

(52) U.S. Cl. 424/490; 514/2; 514/12; 514/23; 514/44 R

(57) ABSTRACT

A system for electrostatically coating particles is provided. The system is particularly well suited for coating charged drug delivery particles (e.g., nanoparticles, microparticles) with a coating of opposite charge. The coating may include a targeting moiety such as a small molecule ligand, peptide, protein, aptamer, etc. The coated particles are biodegradable and/or biocompatible, have a near neutral zeta (ξ) potential, and are stable in serum. The invention also provides pharmaceutical compositions and kits including the inventive coated particles. Methods of preparing and using the inventive particles are also included.

### RELATED APPLICATIONS

The present application claims priority under 35 U.S.C. §119(e) to U.S. provisional patent application, U.S. Ser. No. 60/893,703, filed Mar. 8, 2007, which is incorporated herein by reference.

### GOVERNMENT SUPPORT

This invention was made with U.S. Government support under Grant EB 00244 awarded by the National Institutes of Health. The U.S. Government has certain rights in the invention.

### BACKGROUND OF THE INVENTION

Gene therapy has the potential to treat many disease including cancer, cardiovascular diseases, metabolic diseases, and autoimmune diseases, but it is currently limited by the inability to delivery nucleic acid-based drugs in a safe and effective manner (Anderson Nature 392(Suppl.):25-30, 1996; Friedman Nature Med. 2:144-147, 1996; Crystal Science 270:404-410, 1995; Mulligan Science 260:926-932, 1993; each of which is incorporated herein by reference). Delivering polynucleotides specifically to targeted cells and/or tissues is particularly challenging.

Thus far, the use of modified viruses as gene transfer vectors has generally represented the most clinically successful approach to gene therapy. However, viral delivery is currently plagued by multiple problems including acute toxicity, cellular immune response, oncogenicity due to insertional mutagenesis, limited cargo capacity, resistance to repeated infection, and production and quality control issues (Kay, M. A.; Glorioso, J. C.; Naldini, L. Nat. Med. 2001, 7, 33-40; Merdan, T.; Kopecek, J.; Kissel, T. Adv. Drug Delivery Rev. 2002, 54, 715-758; each of which is incorporated herein by reference) (for leading references, see: Luo et al. Nat. Biotechnol. 18:33-37, 2000; Behr Acc. Chem. Res. 26:274-278, 1993; each of which is incorporated herein by reference).

Current alternatives to viral vectors include polymeric delivery systems (Zauner et al. Adv. Drug Del. Rev. 30:97-113, 1998; Kabanov et al. Bioconjugate Chem. 6:7-20, 1995; each of which is incorporated herein by reference), liposomal formulations (Miller Angew. Chem. Int. Ed. 37:1768-1785, 1998; Hope et al. Molecular Membrane Technology 15:1-14, 1998; Deshmukh et al. New J. Chem. 21:113-124, 1997; each of which is incorporated herein by reference), and “naked” DNA injection protocols (Sanford Trends Biotechnol. 6:288-302, 1988; incorporated herein by reference). While these strategies have yet to achieve the clinical effectiveness of viral vectors, the potential safety, processing, and economic benefits offered by these methods (Anderson Nature 392(Suppl.):25-30, 1996; incorporated herein by reference) have ignited interest in the continued development of non-viral approaches to gene therapy (Boussif et al. Proc. Natl. Acad. Sci. USA 92:7297-7301, 1995; Putnam et al. Macromolecules 32:3658-3662, 1999; Lim et al. J. Am. Chem. Soc. 121:5633-5639, 1999; Gonzalez et al. Bioconjugate Chem. 10:1068-1074, 1999; Kukowska-Latallo et al. Proc. Natl. Acad. Sci. USA 93:4897-4902, 1996; Tang et al. Bioconjugate Chem. 7:703-714, 1996; Haensler et al. Bioconjugate Chem. 4:372-379, 1993; each of which is incorporated herein by reference). Non-viral vectors have been developed using numerous biomaterials including calcium phosphate, cationic lipids, cationic polymers, dendrimers, and cyclodextrins, but although generally safer than viruses, these methods have much lower transfection efficacy (Pack, D. W.; Hoffman, A. S.; Pun, S.; Stayton, P. S. Nat. Rev. Drug Discov. 2005, 4, 581-593; incorporated herein by reference).

Cationic polymers have been widely used as transfection vectors due to the facility with which they condense and protect negatively charged strands of DNA Amine-containing polymers such as poly(lysine) (Zauner et al. Adv. Drug Del. Rev. 30:97-113, 1998; Kabanov et al. Bioconjugate Chem. 6:7-20, 1995; each of which is incorporated herein by reference), poly(ethylene imine) (PEI) (Boussif et al. Proc. Natl. Acad. Sci. USA 92:7297-7301, 1995; incorporated herein by reference), and poly(amidoamine) dendrimers (Kukowska-Latallo et al. Proc. Natl. Acad. Sci. USA 93:4897-4902, 1996; Tang et al. Bioconjugate Chem. 7:703-714, 1996; Haensler et al. Bioconjugate Chem. 4:372-379, 1993; each of which is incorporated herein by reference) are positively-charged at physiological pH, form ion pairs with nucleic acids, and mediate transfection in a variety of cell lines. Despite their common use, however, cationic polymers such as poly(lysine) and PEI can be significantly cytotoxic (Zauner et al. Adv. Drug Del. Rev. 30:97-113, 1998; Deshmukh et al. New J. Chem. 21:113-124, 1997; Choksakulnimitr et al. Controlled Release 34:233-241, 1995; Brazeau et al. Pharm. Res. 15:680-684, 1998; each of which is incorporated herein by reference). As a result, the choice of cationic polymer for a gene transfer application generally requires a trade-off between transfection efficiency and short- and long-term cytotoxicity. Additionally, the long-term biocompatibility of these polymers remains an important issue for use in therapeutic applications in vivo, since several of these polymers are not readily biodegradable (Uhrich Trends Polym. Sci. 5:388-393, 1997; Roberts et al. J. Biomed. Mater. Res. 30:53-65, 1996; each of which is incorporated herein by reference). Recently, a large library of over 2,000 structurally unique poly(beta-amino esters) was prepared using high-thoughput combinatorial techniques (Anderson, D. G.; Lynn, D. M.; Langer, R. Angew. Chem. Int. Ed. 2003, 42, 3153-3158; U.S. Pat. No. 6,998,115, issued Feb. 14, 2006; U.S. Patent Application 2002/0131951, published Sep. 19, 2002; U.S. Patent Application 2004/0071654, published Apr. 15, 2004; each of which is incorporated herein by reference). These polymers have shown considerable promise in delivering drugs both in vitro and in vivo (Anderson, D. G.; Peng, W. D.; Akinc, A.; Hossain, N.; Kohn, A.; Padera, R.; Langer, R.; Sawicki, J. A. Proc. Natl. Acad. Sci. U.S.A. 2004, 101, 16028-16033; Anderson, D. G.; Akinc, A.; Hossain, N.; Langer, R. Mol. Ther. 2005, 11, 426-434; Akinc, A.; Langer, R. Biotechnol. Bioeng. 2002, 78, 503-508; Lynn, D. M.; Langer, R. J. Am. Chem. Soc. 2000, 122, 10761-10768; Lynn, D. M.; Anderson, D. G.; Putnam, D.; Langer, R. J. Am. Chem. Soc. 2001, 123, 8155-8156; each of which is incorporated herein by reference).

A number of techniques for functionalizing particles has been tested. For example, commonly used gene delivery polymers, such as polylysine and polyethylenimine (PEI), have been covalently modified to include poly(ethyleneglycol) (PEG) and/or targeting ligands such as EGF (10; incorporated herein by reference), transferrin (Ogris, M.; Walker, G.; Blessing, T.; Kircheis, R.; Wolschek, M.; Wagner, E. J. Controlled Release 2003, 91, 173-181; incorporated herein by reference), and RGD peptides (Zuber, G.; Dauty, E.; Nothisen, M.; Belguise, P.; Behr, J. P. Adv. Drug Delivery Rev. 2001, 52, 245-253; Kunath, K.; Merdan, T.; Hegener, O.; Haberlein, H.; Kissel, T. J. Gene Med. 2003, 5, 588-599; each of which is incorporated herein by reference) to promote specific uptake. The results of these experiments have bee mixed, with some researchers demonstrating dramatically improved targeting and other showing little improvement (Ogris, M.; Walker, G.; Blessing, T.; Kircheis, R.; Wolschek, M.; Wagner, E. J. Controlled Release 2003, 91, 173-181; Zuber, G.; Dauty, E.; Nothisen, M.; Belguise, P.; Behr, J. P. Adv. Drug Delivery Rev. 2001, 52, 245-253; Kunath, K.; Merdan, T.; Hegener, O.; Haberlein, H.; Kissel, T. J. Gene Med. 2003, 5, 588-599; Kursa et al. Bioconjugate Chem. 2003, 14, 222-231; Thomas, M.; Klibanov, A. M. Appl. Microbiol. Biotechnol. 2003, 62, 27-34; each of which is incorporated herein by reference). Peptides containing the amino acid sequence Arg-Gly-Asp (RGD) can be used for specific targeting to integrin receptors, including the vitronectin receptor αvβ3, which is known to be highly up-regulated in certain tumors (Kunath, K.; Merdan, T.; Hegener, O.; Haberlein, H.; Kissel, T. J. Gene Med. 2003, 5, 588-599; incorporated herein by reference).

One of the problems with covalently coupling targeting ligands to a polymer is that it can change the biophysical properties of that polymer and the corresponding polymer/DNA nanoparticle. For example, several researchers have found that as ligand substitution (either targeting or shielding) increases, overall gene delivery can decrease, presumably due to alteration of the original polymer's functionality for DNA condensation and endosomal buffering (Kursa et al. Bioconjugate Chem. 2003, 14, 222-231; Suh et al. Mol. Ther. 2002, 6, 664-672; each of which is incorporated herein by reference). Furthermore, gene delivery nanoparticles are generall positively charged, and these positively charged nanoparticles are taken up non-specifically (Thomas et al. Appl. Microbiol. Biotechnol. 2003, 62, 27-34; incorporated herein by reference). Cell-specific delivery should include specific uptake by the target cell and reduced delivery to non-targeted cells.

Given the difficulty in targeting specific cells or tissues using polymeric drug delivery systems and having the polymeric drug delivery system taken up by the cell, there remains a need in the art for better designing such drug delivery systems.

### SUMMARY OF THE INVENTION

The present invention provides novel polymeric drug delivery systems of charged polymeric particles coated electrostatically with an oppositely charged coating material, optionally associated with a targeting agent. A positively charged particle is typically coated with a negatively charged coating material, and a negatively charged particle with a positively charged coating material. Coating a charged particle with an oppositely charged coating reduces the net charge on the particle thereby reducing the non-specific uptake of these particles while at the same time facilitating receptor-mediated uptake. In general, neutral or negatively charged coated particles are also less likely to interact with serum proteins. The inventive coated particles also have such favorable biophysical characteristics as biodegradability, small particle size, near-neutral ξ potential, low cytotoxicity, and/or stability in serum. These characteristics make the inventive coated particles particularly useful for delivering bioactive agents such as vaccines, drugs, peptides, proteins, polynucleotides, etc. or diagnostic agents such as radiolabels, labelled compounds, metals, etc. The particles are also particularly useful for targeted delivery of an agent to a cell, tissue, or organ.

In one aspect, the invention provides electrostatically coated polymeric particles. The inventive particles include an agent to be delivered encapsulated in a net positively charged matrix, thereby rendering the particles prior to coating cationic. Any agent that may be delivered using the inventive coated particles include small molecules, polynucleotides, proteins, peptides, etc. The weight to weight (w/w) ratio of polymer to agent ranges from approximately 0.1 w/w to approximately 100 w/w. The ratio of polymer to agent can depend on the polymer and/or agent being used. The cationic particles with their payload are coated with a polyanionic polymer or oligomer optionally associated with a targeting agent (e.g., a peptide, protein, glycopeptide, carbohydrate) or surface modifying agent. Typically, the polyanionic polymer or oligomer is covalently attached to the targeting agent (e.g., an RGD peptide, ligand for a cell surface receptor, aptamer, antibody fragment, etc.) or surface modifying agent, optionally through a linker. The linker may be a cleavable linker. In certain embodiments, the linker may be hydrolyzed, may be cleaved by an enzyme (e.g., an esterase or protease), may be pH-sensitive, may be responsive to the presense or absence of a ligand, may be sensitive to a specific redox potential (e.g., a disulfide linkage), or may be cleaved electrically. The polyanionic coating binds to the cationic particle via electrostatic interactions and also neutralizes at least a portion of the positive charge on the particle. The coating may also optionally include other polymers (e.g., polyethylene glycol (PEG)), small molecules, proteins, peptides, or polynucleotides as surface modifying agents. The coating or polymer of the particle may allow for the “smart” release of the payload from the particle. For example, the payload may be released upon entry into a cell or particular subcellular compartment, or the payload may be released at a certain time. The coated particles typically range in size from approximately 1 nm to 10,000 nm in diameter. In certain embodiments, the particles are approximately 1 nm to 1,000 nm in diameter. In certain embodiments, the particles are approximately 10 nm to 500 nm in diameter. Exemplary cationic polymers useful in preparing the matrix of the particles include polyamines, polylysine, polyhistidine, polyguanine, polyethyleneimine, poly(beta-amino esters), polyamide, and cationic proteins. The anionic polymers used in the coating are typically negatively charged peptides, for example, polyglutamate (e.g., approximately 12-16 residues), polyaspartate (e.g., approximately 12-16 residues), or co-polymers thereof. Any targeting agent be may used in coating the particles including, for example, peptides, proteins, carbohydrates, polynucleotides (e.g., aptamers), small organic molecules, metals, organometallic complexes, polymers, and lipids. In certain embodiments, the inventive coated particles have a near neutral ξ potential (e.g. +10 to −10 mV, or +5 to −5 mV).

Pharmaceutical compositions of the inventive coated particles optionally comprising a pharmaceutically acceptable excipient are also included within the invention. The inventive particles or pharmaceutical compositions may also be included in conveniently packaged kits with other materials and/or instructions for use. As would be appreciated by one of skill in this art, negatively charged particles may be coated using a cationic coating material (e.g., including polyhistidine, polylysine, polyamines, poly(beta-amino esters), etc. optionally associated with a targeting agent or other surface modifying agent.

In another aspect, the invention provides methods of preparing the inventive coated particles described herein. The coated particles are typically prepared by contacting cationic particles encapsulating a payload with anionic polymers associated with a targeting agent. In certain embodiments, the particles to be coated are prepared first and are subsequently coated using a mixture of the anionic coating material. The particles may be prepared using any method known in the art including double emulsion, single emulsion, spray drying, freeze drying, phase inversion, etc. prior to coating. For example, a suspension of particles is combined with a solution of the anionic coating material under suitable conditions to allow the particles to become coated. In certain embodiments, the coating material is a blend of different coating (e.g., a blend of cationic and anionic coating materials). The pH and/or salt concentrations of the solutions and mixtures may be controlled/adjusted to yield efficient coating of the particles and/or yield a desired size of particle. In certain embodiments, the particles and/or the coating material are dissolved in a buffered solution with a pH of approximately 5.0. In other embodiments, the formation of the particles and the coating are done in one pot. For example, the agent to be delivered is mixed with a solution of polymer to form the desired particles and then mixed with the anionic coating material to provide the inventive coated particles. The coating of the cationic particles is typically performed under conditions to yield a nearly neutral coated particle (e.g., a ξ potential of 0 to −5 mV). In order to achieve this, the amount and/or concentrations of particles and coating material are adjusted in the coating step. Anionic particles may also be coated with cationic coating materials using the inventive method.

The invention further provides methods of using the inventive coated particles. The particles may optionally be combined with a pharmaceutically acceptable excipient to form a pharmaceutical composition. The particles or a pharmaceutical composition thereof may be used to deliver an agent to a subject, including a human subject. Therefore, the inventive coated particles may be used to treat or prevent a pathological condition. In certain embodiments, the particles are for a prophylactic use (e.g., to prevent an infection, to prevent pregnancy). The particles may also be used as diagnostic agents, for example, the particles may include a constrast agent or labelled agent for imaging (e.g., CT, NMR, x-ray, ultrasound). The particles or a pharmaceutical composition thereof can be administered to a subject using any available route, for example, oral, parenteral, intravenously, submucosal, subcutaneous, intramuscular, etc. A sufficent amount of the particles is typically administered to achieve the desired result. In certain emodiments, it is an amount sufficient to treat a diease. In other embodiments, it is an amount sufficient to raise blood levels of the agent to be delivered to a desired concentration. As would be appreciated by one of skill in the art, the administration of the inventive coated particles or a pharmaceutical composition thereof including the dose, timing, length of administration, etc. may be determined by a health care professional. The invention also provides kits with the particles or pharmaceutical compositions of the particles for use in the clinic by a medical professional. The kits may include multiple dosage units, devices for administration of the particles, pharmaceutical excipients, and/or instructions for use.

### Definitions

“Animal”: As used herein, the term “animal” refers to any member of the animal kingdom. In some embodiments, “animal” refers to a human, at any stage of development. In some embodiments, “animal” refers to a non-human animal, at any stage of development. In some embodiments, animals include, but are not limited to, mammals, birds, reptiles, amphibians, fish, and/or worms. In certain embodiments, the non-human animal is a mammal (e.g., a rodent, a mouse, a rat, a rabbit, a monkey, a dog, a cat, a sheep, cattle, a primate, and/or a pig). In some embodiments, an animal may be a transgenic animal, genetically-engineered animal, and/or clone.

“Antibody”: The term “antibody” refers to an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In certain embodiments, antibodies of the IgG class are used.

“Antibody fragment”: The term “antibody fragment” refers to any derivative of an antibody which is less than full-length. Examples of antibody fragments include, but are not limited to, Fab, Fab′, F(ab′)₂, scFv, Fv, dsFv diabody, Fc, and Fd fragments. In certain embodiments, the fragment is an Fc fragment, more particularly an Fc fragment of an IgG antibody. The antibody fragment may be produced by any means. For instance, the antibody fragment may be enzymatically or chemically produced by fragmentation of an intact antibody, or it may be recombinantly produced from a gene encoding the partial antibody sequence. Alternatively, the antibody fragment may be wholly or partially synthetically produced. The antibody fragment may optionally be a single chain antibody fragment. A functional antibody fragment will typically comprise at least about 50 amino acids and more typically will comprise at least about 200 amino acids.

“Associated with”: When two entities are “associated with” one another as described herein, they are linked by a direct or indirect covalent or non-covalent interaction. In certain embodiments, the association is covalent. Exemplary covalent associations include carbon-carbon bonds, disulfide bonds, ester bonds, and amide bonds. In other embodiments, the association is non-covalent. Desirable non-covalent interactions include hydrogen bonding, van der Waals interactions, hydrophobic interactions, magnetic interactions, electrostatic interactions, pi stacking, dipole-dipole interactions, ligand-receptor interactions, etc.

“Biocompatible”: The term “biocompatible”, as used herein is intended to describe compounds that are not toxic to cells. Compounds are “biocompatible” if their addition to cells in vitro results in less than 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effects on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed. For example, the inventive materials may be broken down in part by the hydrolysis of the polymeric material of the inventive coated particles.

“Carbohydrate”: The term “carbohydrate” refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbodyhdrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

“Particle”: The term “particle” refers to a small object, fragment, or piece of material and includes, without limitation, polymeric particles, biodegradable particles, non-biodegradable particles, single-emulsion particles, double-emulsion particles, coacervates, liposomes, microparticles, nanoparticles, macroscopic particles, pellets, crystals, aggregates, composites, pulverized, milled or otherwise disrupted matrices, cross-linked protein or polysaccharide particles. Particles may be composed of a single substance or multiple substances. In certain embodiments of the invention the particle is not a viral particle. In other embodiments, the particle is not a liposome. In certain embodiments, the particle is not a micelle. In certain embodiments, the particles is substantially solid throughout.

“Peptide” or “protein”: According to the present invention, a “peptide” or “protein” comprises a string of at least three amino acids linked together by peptide bonds. The terms “protein” and “peptide” may be used interchangeably. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In certain embodiments, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Pharmaceutical agent” or “drug”: “Pharmaceutical agent”, also referred to as a “drug” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Pharmaceutical agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18^{th} ed (2006) following its publication, Mark H. Beers and Robert Berkow (eds.), Merck Publishing Group, or, in the case of animals, The Merck Veterinary Manual, 9^{th} ed., Kahn, C. A. (ed.), Merck Publishing Group, 2005.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. In certain embodiments, the molecular weight is less than 1000 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“Surface modifying agents”: As used herein, the term “surface modifying agent” refers to any chemical compound that can be attached to the surface of a particle using electrostatic coating as described herein. The surface modifying agent may be any type of chemical compound including small molecules, polynculeotides, proteins, peptides, metals, polymers, oligomers, organometallic complexes, lipids, carbohydrates, etc. The agent may modify any property of particle including surface charge, hydrophilicity, hydrophobicity, zeta potential, size, thickness of coating, etc. In certain embodiments, the surface modifying agent is a polymer such as polyethylene glycol (PEG). In certain embodiments, the surface modifying agent is a hydrocarbon.

### BRIEF DESCRIPTION OF THE DRAWING

FIG. 1. Synthesis schemes. (A) Structure and synthesis of poly(beta-amino ester) C32 with the peptide EEEEEEEEEEEEGGGGGGGRGD (SEQ ID NO: X); (B) Electrostatic self-assembly of ligand coated nanoparticles; and (C) transmission electron microscopy of 40 w/w C32 particles coated with 19 w/w E12RGD in serum media. The scale bars are 100 nm in both photomicrographs.

FIG. 2. Ligand coated nanoparticles are slightly larger in size than uncoated nanoparticles. Importantly, coated nanoparticles have a stable, small size in 12% serum-containing media over time. Nanoparticles are formed with 50 w/w C32 and peptide coats with an overall N/P of 1.55. Error bars are standard deviations of independently prepared partcile batches.

FIG. 3. Ligand coated nanoparticles are more neutrally charged in 12% serum-containing media than uncoated nanoparticles. Nanoparticles are formed with 50 w/w C32 and peptide coates with an overall N/P of 1.55. Error bars are standard deviations of independently prepared particle batches.

FIG. 4. FACS results showing the gating of transfected HUVECs. (A) E12-RGD (ligand) coated nanoparticles significantly transfect HUVECs whereas (B) E12-RDG (control) coated nanoparticles do not. Nanoparticles are formed with 40 w/w C32 and overall N/P of 1.55. These results are representative samples from quadruplicate experiments.

FIG. 5. Efficacy and specificity of E12-RGD/E12-RDG ligand coated gene delivery nanoparticles is dependent on w/w peptide and N/P ratio. Nanoparticles are formed at 50 w/w C32 and are delivered to HUVECs in 12% serum containing media. Error bars are standard deviations and (*) and (**) indicate statistical significance of p<0.05 and p<0.01 respectively.

FIG. 6. Efficacy and specificity of E12-RGD/E12-RDG ligand coated gene delivery nanoparticles is dependent on w/w C32 and N/P ratio. Nanoparticles are delivered to HUVECs in 12% serum containing media. Error bars are standard deviations and (*) and (**) indicate statistical significance of p<0.05 and p<0.01 respectively.

FIG. 7. Competition experiment with E12-RGD (ligand) and E12-RDG (control) coated gene delivery nanoparticles and free RGDS peptide fragment. Nanoparticles are formed at 40 w/w C32, N/P=1.55, and are delivered to HUVECs in 12% serum containing media. Error bars are standard deviations and (**) indicates statistical significance of p<0.01.

FIG. 8. Efficacy and specificity of E12-RGD (ligand) and E12-RDG (control) coated gene delivery nanoparticles is dependent on peptide length and N/P ratio. Nanoparticles are formed at 50 w/w C32 and are delivered to HUVECs in 12% serum-containing media. Error bars are standard deviations and (*) and (**) indicate statistical significance of p<0.05 and p<0.01 respectively.

FIG. 9. Electrostatic coating of nanoparticle with E12-PEG-RGD. 50 w/w C32 with various weight ratios of E12-PEG-RGD.

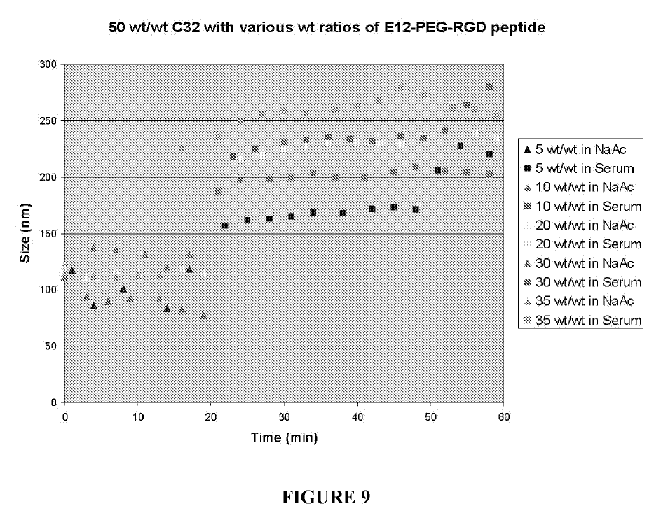

FIG. 10. Particle size of 30 w/w C32 plus E12-PEG vs. media conditions. PEG only (no targeting agent) coated nanoparticles can reduce the particle size in serum vs. uncoated nanoparticles by >50%.

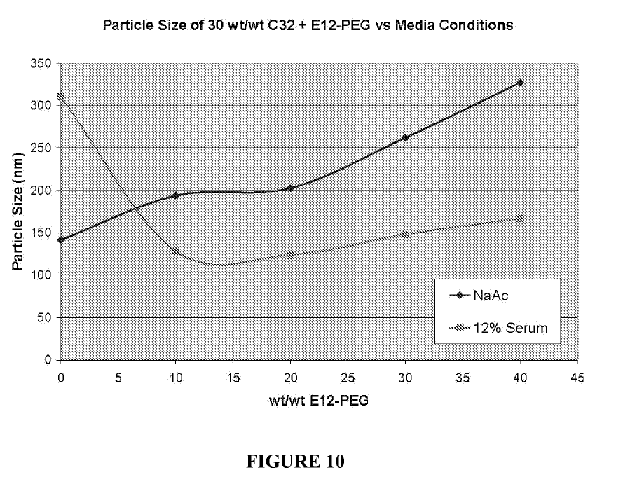

FIG. 11. Electrostatic coating using similarly charged coating. Polylysine-based coatings enhance the overall tranfection efficacy of poly(beta-amino ester)(C32)/DNA (0.02 μg/μl DNA, OptiMem) complexes.

FIG. 12. 50 w/w C32 with various weight ratios of K8-PEG-RGD peptide. In serum, weight ratios of 0-1 w/w K8-PEG-RGD reduce the overall particle size whereas weight ratios 5-14 w/w increase particle size.

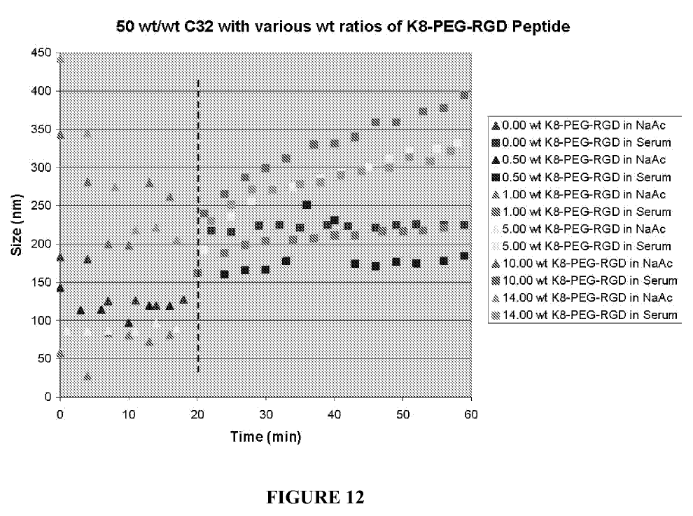

FIG. 13. C32-7/E12-RGD/K8-PEG-RGD in NaAc buffer and serum-containing media. Blends of cationic and anionic coats enable control over nanoparticle size and stability to size much smaller and/or much larger than what is able with single coats. Different biophysical properties are seen when these particles are switched from buffer to serum-containing media (at 20 minutes).

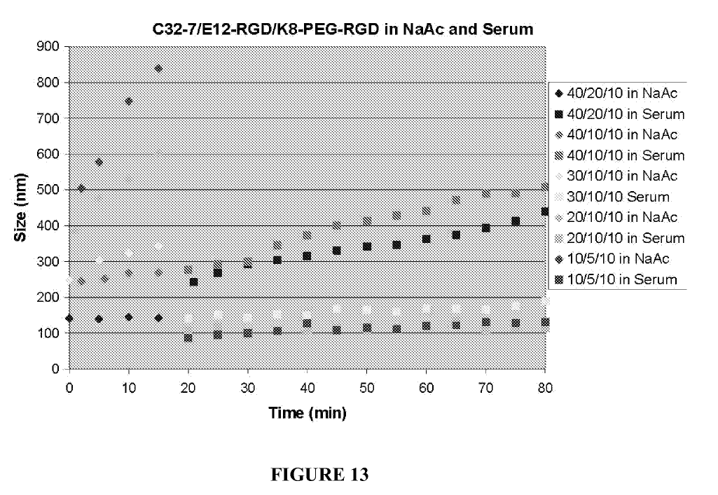

FIG. 14. Cytotoxity of composition on cells using a cytotoxic drug itself as the coating. 70 w/w C32 particles with 600 ng DNA were coated with 100 nM gelonin (a 28 kD protein) to test the cytotoxicity of the compostion in LS174T cells.

FIG. 15 shows the uptake of DNA encoding GFP by C32 particles electrostatically coated with different ligands (cRGD, cCAQ, cCGN, cCAR, Fshort, and Flong). The percentage of cells expressing GFP as determined by flow cytometry are shown. A one-way ANOVA followed by Dunnett's post-test was used to compare each group of ligand-coated particles (black bars) to uncoated control particles (white bar). Data is reported as means plus standard deviation. *p<0.05; **p<0.01. See Example 5.

### DETAILED DESCRIPTION OF CERTAIN EMBODIMENTS OF THE INVENTION

The present invention provides a novel drug delivery system of electrostatically coated particles. The electrostatic coating is used to control various biophysical characterstics of the particles including particle size, surface charge, interaction with serum proteins, cellular uptake, etc. In certain embodiments, the inventive particles have favorable biophysical characterstics including small particle size, near-neutral ξ potential, and stability in serum. Electrostatic coating of particles also offers a convenient way of adding a targeting agent or other surface modifying agent to the surface of a particle. The invention thereby allows for effective ligand-specific targeting of particular cells and/or tissues. The coated particle have been shown to be particularly useful in delivering polynucleotides to targeted cells (e.g., endothelial cells, cancer cells). The inventive system for coating particles for drug delivery also may be used for coating the surface of a particle with surface modifying agents, such as pharmaceutical agents, hydrophilic or hydrophobic substances, polymers, etc. The inventive drug delivery system has several advantages over existing technology, incuding high efficacy, ligand-based specificity, biocompatiblity, biodegradability, and low cytotoxicity. Therefore, the inventive system, which includes pharmaceutical compositions of the inventive electrostatically coated particles, is particularly useful in clinical applications—both diagnostic and therapeutic applications.

### Particles for Coating

The particles suitable for electrostatic coating typically possess a net charge. The particles are cationic or anionic in nature. For example, the particle has a net positive charge when the particle is created from an encapsulating cationic polymer. In certain embodiments, the positive charge imparted by the encapsulating polymer is greater than the negative charge of the payload such as a polynucleotide. In certain embodiments, the particles before coating have a zeta (ξ) potential ranging from +1 to +25 mV. In certain embodiments, the particles have a zeta potential ranging from +5 to +20 mV. In other embodiments, the particles have a zeta potential ranging from +5 to +15 mV. Negatively charged particles may also be coated based on the invention with a positively charged coating material. In certain embodiments, the particles before coating have a zeta (ξ) potential ranging from −1 to −25 mV. In certain embodiments, the particles have a zeta potential ranging from −5 to −20 mV. In other embodiments, the particles have a zeta potential ranging from −5 to −15 mV. The zeta potential of the particles may be measured using any method known in the art and using any environment. In certain embodiments, the measurement of zeta potential is done in water. In other embodiments, the measurement is done in a solution more typical of a physiological environment. In certain embodiments, the measurement is done in culture medium which may optionally include serum. In certain particular embodiments, the measurement is done in 12% serum-containing medium.

A particle of any size may be coated ranging from picoparticles up to microparticles. In certain embodiments, the particle is a picoparticle. In other embodiments, the particle is a nanoparticle. In other embodiments, the particle is a microparticle. In certain embodiments, the average diameter of the particles ranges from approximately 1 μm to 1 μm. In certain embodiments, the average diameter of the particles range from approximately 1 nm to approximately 100 μm. In certain embodiments, the particles range in size from 10 nm to 500 nm. In other embodiments, the particles range in size from 100 nm to 500 nm. In certain embodiments, the particles range in size from 500 nm to 1,000. In other embodiments, the particles range in size from 1,000 nm to 5,000 nm. In other embodiments, the particles range in size from 1,000 nm to 2,500 nm. In other embodiments, the particles range in size from 2,500 nm to 5,000 nm. In certain embodiments, the particles range in size from 1 nm to 100 nm. In certain embodiments, the particles range in size from 100 nm to 200 nm. In certain embodiments, the particles range in size from 200 nm to 300 nm. In certain embodiments, the particles range in size from 300 nm to 400 nm. In certain embodiments, the particles range in size from 400 nm to 500 nm. In certain embodiments, the particles range in size from 500 nm to 600 nm. In certain embodiments, the particles range in size from 600 nm to 700 nm. In certain embodiments, the particles range in size from 700 nm to 800 nm. In certain embodiments, the particles range in size from 800 nm to 900 nm. In certain embodiments, the particles range in size from 900 nm to 1,000 nm. The measurments described herein typically represent the average particle size of a population. However, in certain embodiments, the measurements may represent the range of sizes found in a population, or the maximum or minimum size of particles found in the population.

The particles to be coated typically comprise an agent to be delivered, such as a pharmaceutical agent, encapsulated in a matrix. The agent may be distributed throughout the particle, the agent may be found on the surface of the particle, and/or the agent may be found in the core of the particle. In addition to pharmaceutical agents, diagnostic agents, contrast agents, prophylactic agents, nutrients, etc. can be the payload of the inventive particles. In certain embodiments, the particle is a liposome. In other embodiments, the particle is substantially solid and does not include a gaseous or liquid core. In certain embodiments, the particle is not a liposome. In certain embodiments, the particle is a micelle. In certain embodiments, the particle is not a micelle.

The particles to be coated can be prepared from any material (e.g., protein, carbohydrates, lipids, polymers, metal, ceramics, etc.). Typically, the particles are prepared using polymeric materials. The polymers of the matrix typically have charged moieties that are present on the surface of the particle. Such moieties facilitate electrostatically coating the outside of the particle with a charged material (e.g., an oppositely charged peptide). The polymer may be a natural polymer or a synthetic polymer. The polymers may be straight chain polymers, branched polymers, dendritic polymers, co-polymers, block polymers, or cross-linked polymers. In preparing positively charged particles, a polymer with a net positive charge is typically used. Cationic polymers useful in the invention include polymers that contain amino moieties (e.g., primary, secondary, tertiary, or quaternary amines). Positively charged salts of polymers may also be used to prepare the particles. Anionic polymers useful in the invention include polymers that contain carboxylic acid, sulfate, or phosphate moieties. Negatively charged salts of polymers may also be used to prepare the particles. Exemplary polymers useful in the present invention include polyamines, polyethers, polyamides, polyesters, poly(beta-amino esters), polycarbamates, polyureas, polycarbonates, poly(styrene) derivatives, polyimides, polysulfones, polyurethanes, polyacetylenes, polyethylenes, polyethyeneimines, polyisocyanates, polyacrylates, polymethacrylates, polyacrylonitriles, and polyarylates. In certain embodiments, the polymer is a natural polymer, for example, polypeptides, polysaccharides, and polynucleotides. In certain embodiments, the polymer is poly(lysine). In other embodiments, the polymer is polyethyleneimine (PEI). In other embodiments, the polymer is a poly(beta-amino ester). Various poly(beta-amino esters) are described in U.S. Pat. No. 6,998,115, issued Feb. 14, 2006; U.S. patent application 2005/0265961, published Dec. 1, 2005; and U.S. patent application 2004/0071654, published Apr. 15, 2004; each of which is incorporated herein by reference. In certain embodiments, the polymer is a positively charged protein. In certain embodiments, the polymer is a negatively charged protein. The matrix of the particle may include multiple polymers. The polymers may be a mixture of polymers or be co-polymers. In certain embodiments, the polymers are cross-linked.

The particle may be composed of other materials besides polymers. In certain embodiments, a ceramic such as calcium phosphate ceramic is used. Exemplary calcium phosphate ceramics include tricalcium phosphate, hydroxyapatite, and biphasic calcium phosphate. The particles may be composed of inorganic material such as zeolite. In certain embodiments, the particles comprise a carbohydrate. In certain embodiments, the particles comprise a lipid. In certain embodiments, the particles comprise a protein or peptide.

The agent to be delivered can be any chemical compound. Exemplary chemical compounds include small organic molecules, polynucleotides, proteins, peptides, carbohydrates, polymers, metals, and organometallic complexes. In certain embodiments, the agent to be delivered is a small organic molecule (e.g., a small molecule drug). In other embodiments, the agent is DNA. In other embodiments, the agent is RNA. In other embodiments, the agent is an siRNA agent (e.g. shRNA). In certain embodiments, the agent is a peptide. In other embodiments, the agent is a protein. Classes of agent that may be administered using the inventive particles include pharmaceutical agents, prophylactic agents, nutrients, diagnostic agents, etc. In certain embodiments, the pharmaceutical agent is a drug approved by the U.S. Food and Drug Administration for use in humans. In other embodiments, the agent is a prophylactic agent such as a vaccine or agent to prevent pregnancy. In other embodiments, the agent is a nutritional supplement, a vitamin, or a mineral. In yet other embodiments, the agent is a diagnostic agent such as a contrast agent for imaging.

The particle may be prepared using any techniques known in the art. Exemplary methods of preparing particles include freeze drying, spray drying, double emulsion, single emulsion, phase inversion, etc. In certain embodiments, the particles are preapred by spray drying. The particles for coating may also be purchased or provided by a third party.

The particle may include 0.1% to 99% by weight of the agent to be delivered. In certain embodiments, the particle includes approximately 0.5% to 80% by weight of the agent to be delivered. In certain embodiments, the particle includes approximately 20% to 70% by weight of the agent to be delivered. In certain embodiments, the particles includes 1% to 30% by weight of the agent to be delivered. In certain embodiments, the particles includes 1-10% by weight of the agent. In certain embodiments, the particles includes 10-20% by weight of the agent. In certain embodiments, the particles includes 20-30% by weight of the agent. In certain embodiments, the particles includes 30-40% by weight of the agent. In certain embodiments, the particles includes 40-50% by weight of the agent. In certain embodiments, the particles includes 50-60% by weight of the agent. In certain embodiments, the particles includes 60-70% by weight of the agent. In certain embodiments, the particles includes 70-80% by weight of the agent. In certain embodiments, the particles includes 80-90% by weight of the agent. In certain embodiments, the particles includes 90-99% by weight of the agent. In certain particular embodiments, the particles include approximately 1%, 2%, 3%, 4%, 5%, 6%, 7%, 8%, 9%, or 10% by weight agent. In other embodiments, the particles include approximately 15%, 20%, 25%, 30%, 35%, 40%, 45%, 50%, 55%, 60%, 65%, 70%, 75%, 80%, 85%, 90%, or 95% by weight agent. As will be appreciated by one of skill in the art, the amount of agent in the particle can be adjusted depending on the agent to be delivered. More potent agents are typically included in the particles at a lower percetage than less potent agents.

### Coating

The particle are coated with a charged material. In certain embodiments, the particles are coated with a material of opposite charge. However, in certain embodiments, the particles are coated with a blend of material with oppositve charge as well as similar charge. In certain embodiments, if the particles carry a positive charge, the coating typically is negatively charged. If the particles are negatively chared, the coating is positively charged. The coating may include any type of material. In certain embodiments, the coating includes a polymeric or oligomeric component. In certain embodiments, the coating includes a peptide or protein. In certain embodiments, the coating includes a charged oligomer (e.g., anionic oligomer or cationic oligomer). In other embodiments, the coating includes a charged polymer (e.g., anionic polymer, or ca tionic polymer). The coating material is optionally associated with a targeting agent or surface modifying agent. The coating material may also include a linker between the targeting agent/surface modifying agent and the charged component. The linker is thought to allow the targeting agent more freedom in interacting with its target on a cell. The coating material may also include an agent that changes the surface characteristics of the particle (e.g., polyethylene glycol). The coating material may also include an agent to be delivered such as a pharmaceutical agent.

In certain embodiments, the coating material is negatively charged and is used to coat a positively charged particle. The coating material typically includes chemical functional groups that possess a negative charge under physiological conditions (e.g., pH ˜7.4). In certain embodiments, the anionic functional group is a carboxylic acid moiety. In other embodiments, the anionic functional group is a phosphate. In other embodiments, the anionic functional group is a phosphonate. In other embodiments, the anionic functional group is a sulfate. In certain embodiments, the anionic functional group is a sulfonic acid. In certain other embodiments, the negatively charged functional group is negatively charged at a pH greater than 7.5 or 8.0. In certain embodiments, the negatively charged functional group is an alkoxide. In certain embodiments, it is a phenoxide. The negatively charged component includes 1 to 50 negative charges at physiological pH. In certain embodiments, the number of negative charges ranges from 5 to 25. In more specific embodiments, the number of negative charges ranges from 10 to 20. In particular embodiments, the number of negative charges is 12, 13, 14, 15, or 16. In certain embodiments, the number of negative charges is less than 10.

In certain embodiments, the negatively charged component is a peptide or protein with aspartate or glutamate residues. The peptide or proteins may optionally include other amino acids which may or may not be negatively charged at physiological pH. In certain embodiments, the peptide or protein only includes negatively charged or neutral amino acids at physiological pH. In other embodiments, the peptide or protein contain positively charged amino acids at physiological pH, but the net charge on the peptide or protein is negative. In certain particular embodiments, the peptide is polyglutamate. In other particular embodiments, the peptide is polyaspartate. In other embodiments, the peptide is a co-polymer of polyglutamate and polyaspartate. In certain embodiments, the peptide comprises 1 to 50 glutamate and/or aspartate residues. In certain embodiments, the peptide comprises 5 to 25 glutamate and/or aspartate residues. In certain embodiments, the peptide comprises 10 to 20 glutamate and/or aspartate residues. In other embodiments, the peptide comprises 12 to 16 glutamate and/or aspartate residues. In certain embodiments, the peptide comprises 12, 13, 14, 15, or 16 glutamate and/or aspartate residues.

In certain embodiments, the negatively charged component is a carbohydrate. The carbohydrate may include negatively charged groups such as sulfates or phosphates. In other embodiments, the negatively charged component is a small molecule. In yet other embodiments, the negatively charged component is a polynucleotide. In certain embodiments, the negatively charged component is a synthetic polymer or oligomer. In certain particular embodiments, the negatively charged component is a synthetic polymer or oligomer including carboxylic acid moieities. In certain particular embodiments, the negatively charged component is a synthetic polymer or oligomer including phosphate or sulfate moieities.

In certain embodiments, the coating material is positively charged and is used to coat a negatively charged particle. The coating material typically includes chemical functional groups that possess a positive charge under physiological conditions (e.g., pH ˜7.4). In certain embodiments, the cationic functional group is a nitrogen-containing moiety (e.g., an amine, imidazolyl, guanidine, etc.). In certain other embodiments, the positively charged functional group is positively charged at a pH less than 7.4 or 7.0. The positively charged component includes 1 to 50 positive charges at physiological pH. In certain embodiments, the number of positive charges ranges from 5 to 25. In more specific embodiments, the number of positive charges ranges from 10 to 20. In particular embodiments, the number of positive charges is 12, 13, 14, 15, or 16. In certain embodiments, the number of positive charges is less than 10.

In certain embodiments, the positively charged component is a peptide or protein with lysine, histidine, or arginine residues. The peptide or proteins may optionally include other amino acids which may or may not be positively charged at physiological pH. In certain embodiments, the peptide or protein only includes positively charged or neutral amino acids at physiological pH. In other embodiments, the peptide or protein contain negatively charged amino acids at physiological pH, but the net charge on the peptide or protein is positive. In certain particular embodiments, the peptide is polylysine. In other particular embodiments, the peptide is polyhistidine. In yet other embodiments, the peptide is polyarginine. In other embodiments, the peptide is a co-polymer of polylysine, polyhistidine, and/or polyarginine. In certain embodiments, the peptide comprises 1 to 50 lysine, arginine, and/or histidine residues. In certain embodiments, the peptide comprises 5 to 25 lysine, arginine, and/or histidine residues. In certain embodiments, the peptide comprises 10 to 20 lysine, arginine, and/or histidine residues. In other embodiments, the peptide comprises 12 to 16 lysine, arginine, and/or histidine residues. In certain embodiments, the peptide comprises 12, 13, 14, 15, or 16 lysine, arginine, and/or histidine residues.

In certain embodiments, the positively charged component is a carbohydrate. The carbohydrate may include positively charged groups such as amines. In other embodiments, the positively charged component is a small molecule. In certain embodiments, the positively charged component is a synthetic polymer or oligomer. In certain particular embodiments, the positively charged component is a synthetic polymer or oligomer including amine moieities.

The coating material may optionally include a targeting agent since it is often desirable to target a particular cell, collection of cells, tissue, or organ system. Any targeting agent known in the art of drug delivery may be used in the coating. A variety of targeting agents that direct pharmaceutical compositions to particular cells are known in the art (see, for example, Cotten et al. Methods Enzym. 217:618, 1993; incorporated herein by reference). The targeting agent may be used to target specific cells or tissues (e.g., cancer) or may be used to promote endocytosis or phagocytosis of the coated particle. Electrostatic coating of particles provides for convenient coating of the outside of a particle with a desired targeting agent. A batch of particles may be prepared, and then portion of the particles may be coated with different targeting agents for targeting different cells or tissues. Classes of targeting agents useful in the inventive particles include proteins, peptides, polynucleotides, small organic molecules, metals, metal complexes, carbohydrates, lipids, etc. In certain embodiments, the targeting agent is a protein or peptide. Antibodies (e.g., humanized monoclonal antibody) or antibody fragment (e.g., Fab fragment) may be used as targeting agents. In certain embodiments, a protein receptor or a portion of a protein receptor is used as the targeting agent. In other embodiments, a peptide ligand (e.g. peptide hormone, signaling peptide, peptide ligand, etc.) is used as the targeting agent. In certain particular embodiments, the targeting agent is an RGD integrin-binding peptide. In certain embodiments, a peptide aptamer is used. In certain embodiments, the targeting agent is a glycopeptide or glycoprotein. In certain embodiments, the targeting agent is an avimer. In certain embodiments, the targeting agent is a nanobody. In certain embodiments, the targeting agent is a polynucleotide. In certain particular embodiments, the targeting agent is DNA-based. In other embodiments, the targeting agent is RNA-based. In certain embodiments, the targeting agent is a polynucleotide aptamer. In certain embodiments, the targeting agent is a carbohydrate. In certain embodiments, the targeting agent is a carbohydrate ligand. In certain embodiments, the targeting agent is a carbohydrate found on the surface of a cell. In certain embodiments, the targeting agent is small molecule. In certain embodiments, the targeting agent is an organic small molecule. In other embodiments, the targeting agent is an amino acid. In certain embodiments, the targeting agent comprises a metal. In certain embodiments, the targeting agent is an organometallic complex. Examples of targeting agents include, but are not limited to, low-density lipoproteins (LDLs), transferrin, asialycoproteins, gp120 envelope protein of the human immunodeficiency virus (HIV), sialic acid, RGD-containing peptides, CAQ-containing peptides, CGN-containing peptides, CAR-containing peptides, etc. The targeting agent is associated with the charged portion of the coating material. In certain embodiments, the association is a covalent interaction. Any covalent bond may be used to join the charged portion to the targeting agent. In certain embodiments, a carbon-carbon bond is used. In other embodiments, an ether, ester, amide, disulfide, carbonate, urea, thioether, amine or other heteroatom-heteroatom or heteroatom-carbon atom bond is used. In other embodiments, the association is a non-covalent interaction. Exemplary non-covalent interactions include hydrogen bonding, hydrophobic interactions, van der Waals interactions, dipole-dipole interactions, etc. In certain embodiments, a metal-chelator type interactions is used. In other embodiments, a receptor-ligand interactions is used. In certain embodiments, an antigen-antibody interaction is used. In certain embodiments, a streptavidin-biotin interaction is used.

In certain embodiments, the targeting agent is associated with the charged portion of the coating material through a linker. Any linker known in the art may be used. The linker is provide to allow for freedom of motion of the targeting agent. Preferably, the linker and its attachment to the targeting moiety does not interfere with targeting moiety's interaction with it ligand on the targeted cell. In certain embodiments, the linker is 1 to 50 atoms in length. In certain embodiments, the linker is 5 to 30 atoms in length. In certain embodiments, the linker is 10 to 25 atoms in length. In certain embodiments, the linker is 20 to 30 atoms in length. In certain embodiments, the linker is a peptide. In certain embodiments, the linker comprises polyglycine. In certain embodiments, the linker comprises polyalanine. In other embodiments, the linker is an aliphatic or heteroaliphatic linker. In certain particular embodiments, the linker is an unbranched, unsubstituted alkyl chain. In other embodiments, the linker is an unbranched, unsubstituted heteroaliphatic chain (e.g., containg oxygen, sulfur, or nitrogen atoms). In certain particular embodiments, the linker is a polyethylene glycol linker. In certain embodiments, the linker is cleavable. The linker may be hydrolyzable. In certain embodiments, the linker is pH sensitive. In certain embodiments, the linker is cleaved by an enzyme (e.g., esterase, protease). In certain embodiments, the linker is redox sensitive (e.g., a disulfide bond). The linker is associated with the charged portion of the coating material and is associated with the targeting agent of the coating material. In certain embodiments, a covalent attachment is used. Any covalent bond may be used to join the targeting agent, linker, and charged component (e.g., carbon-carbon bonds, esters, amides, disulfides, ethers, etc.). In certain embodiments, the association is non-covalent (e.g., hydrogen bonding, hydrophobic interactions, dipole-dipole interactions, van der Waals interactions, etc.). In certain embodiments, a metal-chelator type interactions is used. In other embodiments, a receptor-ligand interactions is used. In certain embodiments, an antigen-antibody interaction is used. In certain embodiments, a streptavidin-biotin interaction is used.

The coating material may optionally include other chemical compounds that change the surface characteristics of the coated particle (i.e., a surface modifying agent). For example, the coating material may include a polymer. In certain embodiments, the polymer is a peptide or protein. In othere embodiments, the polymer is not a peptide or protein. In certain embodiments, the polymer is a synthetic polymer. Any polymer may be used. In certain embodiments, the polymer is a hydrophobic polymer. In other embodiments, the polymer is a hydrophilic polymer. In certain embodiments, the polymer is polyethylene glycol (PEG). In certain embodiments, the polymer is a hydrophobic C₁-C₅₀ alkyl chain.

The coating material may optionally include an agent to be delivered by the particle. Any of the agents described herein that can be delivered by the particles can also be delivered by including the agent in the coating. The agent in the particle may be the same or different from the agent of the coating. In certain embodiments, the agent of the coating is different than the agent inside the particle. In other embodiments, the agents inside and in the coating are the same. The agent to be delivered in the coating may be a protein or peptide. In certain embodiments, the agent to be delivered in the coating is a small molecule. In certain embodiments, the agent itself is charged and can be used to coat the particles.

The particles may also be coated with multiple layers. Multiple layers are particularly useful when a single coating or a blend of coating materials will not achieve the desired result. For example, each layer may include a different agent to be released at a different time or under different conditions. In certain embodiments, the mutliple layers provide a controlled release of an agent. In certain embodiments, the multiple layers provide a specific dissolution profile of the agent or agents to be delivered. In certain embodiments, each of the multiple coatings relies on electrostatic interactions to adhere the coating to the particle or the previous layer of coating. For example, a postively charged particle may be coated with a negatively charged coating material followed by a positively charged coating material. In certain embodiments, all of the coatings do not depend on electrostatic interactions. The coatings may be applied using any techniques known in the art. As would be appreciated by one of skill in this art, many coatings may be used. In certain embodiments, the particles have 2-10 coatings. In certain particular embodiments, the particles have 2-5 coatings. In certain embodiments, the particles have 2 coatings. In other embodiments, the particles have 3 coatings. Coating the particles with multiple layers allows for control of the size, surface charge, zeta potential, biodegradability, and/or stability of the coated particles.

### Coated Particles

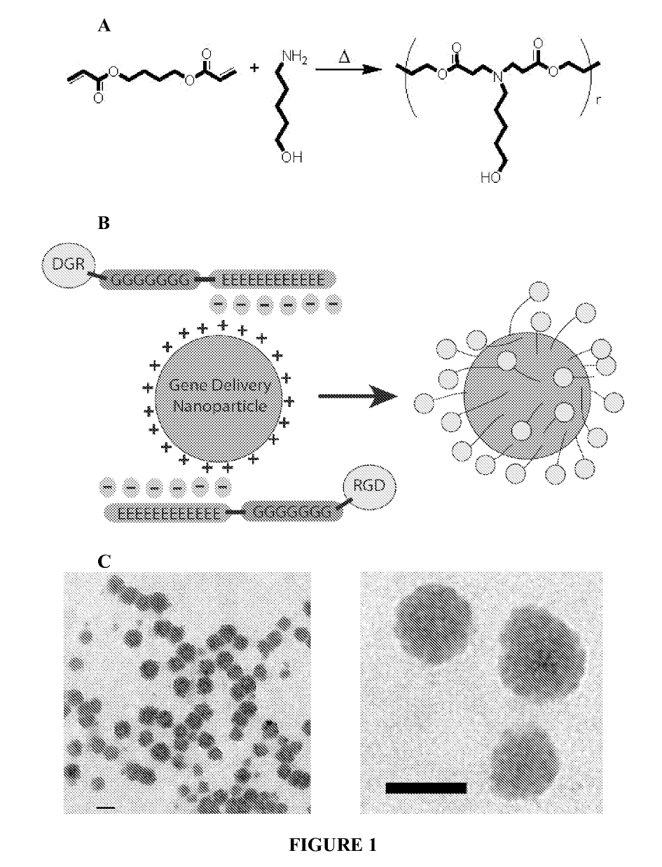

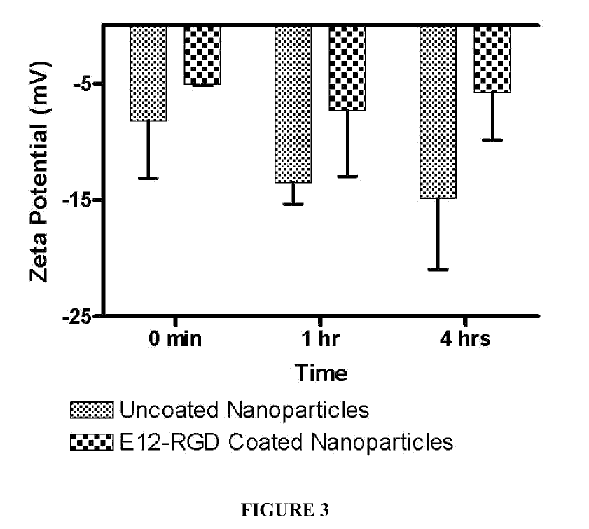

The charged particles are coated with a charged coating material, which optionally includes a targeting moiety, surface modifying agent, or agent to be delivered. The coated particles are electrostatically coated with a charged coating material. In certain embodiments, the charged coating material is one type of compound. In other embodiments, the charged coating material is a mixture of different compounds for coating a particle. An exemplary preparation of an inventive coated particle is shown in FIG. 1. The Examples below also describe various particular methods of preparing the coated particles. In certain embodiments, a positively charged particle is coated with a negatively charged coating material. In certain embodiments, a positively charged particles is coated with a negatively charged coating material which includes a targeting agent. In certain embodiments, a negatively charged particle is coated with a positively charged coating material. In certain embodiments, a negatively charged particles is coated with a positively charged coating material which includes a targeting agent. Any ratio of coating material to particle may be used. In certain embodiments, the coated particle is neutral or near neutral in charge after the coating. In certain particular embodiments, the coated particle is slightly negatively charged. In other particular embodiments, the coated particle is slightly positively charged. The coated particles are typically more neutrally charged in a test medium (such as 12% serum media) than the uncoated particles. In certain embodiments, the zeta potential of the coated particles in 12% serum media ranges from approximately 0 mV to approximately −15 mV. In certain embodiments, the zeta potential ranges from approximately −2 mV to approximately −10 mV. In other embodiments, the zeta potential of the coated particles in 12% serum media ranges from approximately 0 mV to approximately +15 mV. In certain embodiments, the zeta potential ranges from approximately +2 mV to approximately +10 mV. In certain embodiments, the zeta potential of the coated particles ranges from approximately −10 mV to approximately +10 mV. In certain particular embodiments, the zeta potential of the coated particles ranges from approximately −5 mV to approximately +5 mV. The coating of the particle may reduce the zeta potential of the coated particle by approximately 5-10 mV as compared to the uncoated particle. See, e.g., FIG. 3.

In general, a weight to weight ratio is used to refer to the amount of polymer versus payload in a particle (e.g., polynucleotide, small molecule). As the actual fraction of protonated amines in a cationic polymeric particle is unknown, the charge ratio is reported as a ratio of amine groups on the polymer to phosphate groups on a polynucleotide. In the present invention, the N/P ratio, which is typically used for uncoated particles, is modified to also include the negatively charged groups (e.g., carboxylic acid groups) of the coating, that is:

Charge Ratio ˜N/P Ratio=[N]/([P]+[COO⁻])

where N represents the number of amine groups on the polymer, P represents the number of phosphate groups on the polynucleotide, and COO⁻ represents the number of negatively charged carboxylic acid groups on the coating. This is referred to as the “N/P ratio” or “charge ratio.” The charge ratio may range from approximately 10 to approximately 0.5. In certain embodiments, the ratio ranges from approxminately 2.0 to approximately 1.0. In certain embodiments, the ratio ranges from approximately 1.75 to approximately 1.25. In certain particular embodiments, the ratio is approximately 1.5. In certain particular embodiments, the ratio is approximately 1.55. In certain particular embodiments, the ratio is approximately 1.45. In certain particular embodiments, the ratio is approximately 1.35. As would be appreciated by one of skill in this art, for particles with non-amine containing polymers and/or non-phosphate-containing agents, a more general charge ratio may be used based on the positively charged moieties and negative charged moieties within the particle. In certain embodiments, the agent being delivered may be neutral, and the charge of the agent would fall out of the charge ratio calculation.

The coated particles range in size from approximately 1 nm to approximately 100 μm in diameter. In certain embodiments, the coated particles range in size from approximately 10 nm to approximately 1000 nm in diameter. In certain embodiments, the coated particles range in size from approximately 50 nm to approximately 500 nm in diameter. In certain embodiments, the coated particles range in size from approximately 100 nm to approximately 350 nm in diamter. In certain particular embodiments, the coated particles have an average diameter of approximately 50 nm, approximately 100 nm, approximately 150 nm, approximately 200 nm, approximately 250 nm, approximately 300 nm, approximately 350 nm, approximately 400 nm, approximately 450 nm, or approximately 500 nm. The thickness of the coating may be controlled by the coating conditions or the coating material used to yield a coated particle with the desired characteristics (e.g., size, zeta potential, biodegradability, stability, etc.).

The coated particles are typically biodegradable. The particle may degrade over hours to days to weeks to months, thereby releasing its payload over an extended period of time. In certain embodiments, the composition of the coated particle is chosen or adjusted to achieve the desired half-life. In certain embodiments, the half-life of the particle under physiological conditions is 1-24 hours. In certain embodiments, the half-life of the particle under physiological conditions is 1-7 days. In other embodiments, the half-life is from 2-4 weeks. In other embodiments, the half-life is approximately 1 month. In other embodiments, the half-life is 2-3 months. In still other embodiments, the half-life is 4-6 months. In yet other embodiments, the half-life is 6-8 months. In other embodiments, the half-life is 12 months.

The coated particles are particularly useful for administering a therapeutic agent to a subject in need thereof. The coated particles may be administered in any way known in the art of drug delivery, for example, orally, parenterally, intravenously, intramuscularly, subcutaneously, intradermally, transdermally, intrathecally, submucosally, sublingually, rectally, vaginally, etc. A particle coated with a targeting agent has the ability to specifically target a particular cells or tissue in the subject. In certain embodiments, the coated particles are used to specifically target a particular organ (e.g., the liver, GI tract, kidneys, brain, etc.). In certain embodiments, the coated particles are used to target endothelial cells as described in the Examples below. Using an RGD peptide in the coating, the particles are able to specifically target and transfect endothelial cells. In the Examples, human umbilical vein endothelial cells (HUVECs) were used as the model system. Even difficult to transfect HUVECs were transfected using the inventive coated particles. Endothelial cells are a particularly useful target for anti-angiogenesis agents in the treatment of cancer or other proliferative diseases and for anti-atherosclerosis agents in the treatment of cardiovascular disease.

The coated particles are also useful in targeting diseased cells, tissues, or organs. In certain embodiments, the coated particles are used to specifically target a particular diseased tissue. In certain particular embodiments, the coated particles are used to specifically target a cancer in the subject's body. In certain embodiments, the coated particles are used to target an atherosclerotic lesion in a subject. The coated particles are also useful in the treatment of infectious diseases. The coated particles may include a targeting agent to specifically target a microorganism such as a bacteria, fungus, etc. In certain embodiments, the particles are used to target a parasite.

In certain embodiments, one or more of the components of the inventive particles (e.g., coating, matrix) are designed to degrade and/or release their payload at a certain time or when the particles reach a particular location. The component(s) and/or particles may be degraded by a hydrolytic process. Such a process may be pH sensitive. The component(s) and/or particles may be degraded by a process catalyzed by an enzyme. In certain embodiments, a lipase, protease, esterase, etc. catalyzes the break down of at least one component of the inventive particle. In certain embodiments, the particle is senstive to particular redox conditions. For example, a components may be broken down in a reductive or oxdiative environment. In certain embodiments, the particle is degrades depending on the presence, absence, or concentration of a ligand such as a small molecule. Particles that are sensitive to their environment facilitate the “smart” release of the particle's payload allowing for the release of the payload at a particular time or under specific conditions (e.g., in a cell or particular subcellular compartment).

### Preparation of the Coated Particles

Particles are coated electrostatically by contacting a charged particle with a charged coating material. Any methods or techniques known in the art for coating particles may be used. In certain embodiments, the particles to be coated are prepared separately or are obtained from another source such as a commercial source. In certain embodiments, the particle are prepared and then coated in a seemingly continuous process. The particles may be washed, purified, sized, characterized, etc. before the coating process. The contacting is typically done in a suspension or mixture of the particles. In certain embodiments, the medium in which the coating is performed is buffered so that the particles are charged and the coating material is charged. For example, the pH of coating conditions may range from approximately 4.0 to approximately 10.0. In certain embodiments, the pH is approximately 4.5, approximately 5.0, approximately 5.5, approximately 6.0, approximately 6.5, approximately 7.0, approximately 7.5, approximately 8.0, approximately 8.5, approximately 9.0, or approximately 9.5. In certain particular embodiments, an acetate buffer at pH ˜5 is used for the coating process. In certain embodiments, the coating is done in an organic solvent. Examplary organic solvent useful in the process include DMSO, DMF, THF, ethers, glyme, chloroform, carbon tetrachloride, methylene chloride, benzene, toluene, etc. In certain embodiments, a non-halogenated solvent is used in the coating process. A solution of the coating material (at a concentration of 0.1 M to 0.001 M) is mixed with a suspension of the particles to be coated. The resulting mixture is incubated for 1 minute to 4 hours at a temperature ranging from 0° C. to 40° C. In certain embodiments, the coating process takes place over 1-30 minutes, more specifically 1-10 minutes. In certain embodiments, the coating process takes place over approximately 5 minutes. The coating is typically done at room temperature. In certain embodiments, the concentration of the coating material ranges from 10 mM to 50 mM. In a particular embodiments, the concentration of the coating material is approximately 25 mM. As would be appreciated by one of skill in this art, the coating conditions may be varied or optimized depending on the particles to be coated and the coating material to be used. The conditions of the coating process may also depend on the quantity of particles being coated. A larger-scale process may require different conditions than a small-scale process.

After the coated particles are prepared, they may be optionally purified, washed, or sized. In certain embodiments, a sample of the particle is taken for characterization. The characterization may include determining whether the coated particles meet the desired characteristics of the particles (e.g., zeta potential, size, dissolution profile, biodegradability, targeting, etc.). The particles may then be used to prepare a pharmaceutical composition or used as is.

### Pharmaceutical Compositions

Once the inventive coated particles have been prepared, they may be combined with pharmaceutically acceptable excipients to form a pharmaceutical composition. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the coated particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive particles with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the particles in a proper medium. Absorption enhancers can also be used to increase the flux of the compound across the skin. The rate can be controlled by either providing a rate controlling membrane or by dispersing the particles in a polymer matrix or gel.

### Kits

The invention also provides kits for use in preparing or administering the inventive coated particles. A kit for coating particles may include the coating material as well as any solvents, solutions, buffer agents, acids, bases, salts, targeting agent, etc. needed in the coating process. Different kits may be available for different targeting agents. In certain embodiments, the kit include materials or reagents for purifying, sizing, and/or characterizing the resulting coated particles. The kit may also include instructions on how to use the materials in the kit. The particles to be coated are typically provided by the user of the kit.

Kits are also provided for using the inventive coated particles or pharmaceutical compositions thereof. The particles may be provided in convenient dosage units for administration to a subject. The kit may include mutliple dosage units. For example, the kit may include 1-100 dosage units. In certain embodiments, the kit includes a week supply of dosage units, or a month supply of the dosage units. In certain embodiments, the kit includes an even longer supply of dosage units. The kits may also include devices for administering the particles or a pharmaceutical composition thereof. Exemplary devices include syringes, spoons, measuring devices, etc. The kit may optionally include instructions for administering the inventive particles (e.g., prescribing information).

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Electrostatic Ligand Coatings of Nanoparticles for Nucleic Acid Delivery

Coatings that reduce the positive charge of gene delivery nanoparticles could potentially reduce non-specific uptake while still enabling receptor-mediated uptake. Gene delivery nanoparticles at overall neutral or negative charge may also be desirable to prevent unwanted serum interactions.

Here, we show that electrostatic interactions can drive peptide coating of nanoparticles and enable ligand-specific gene delivery to human primary cells. Our general approach to electrostatically coat gene delivery nanoparticles with ligands provides a simple method of ligand addition as well as a mechanism to neutralize nanoparticle charge and reduce electrostatic interactions with undesirable cell types. While we use RGD-containing peptide as a model system to investigate nanoparticle coating and ligand-specific delivery to primary endothelial cells, many other peptide ligand sequences, such as those made from antibody fragments, could potentially be readily incorporated into this system as well.

Nanoparticle Formation and Peptide Coating. Polymer C32 is synthesized by the conjugate addition of 5-aminopentanol to 1,4-butanediol diacrylate. The acrylate and amine monomers used in this experiment and their synthesis scheme can be seen in FIG. 1A.

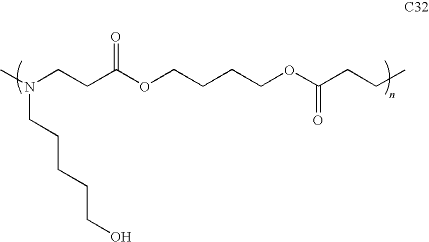

Cationic polymeric gene delivery nanoparticles were formed in sodium acetate buffer solution through self-assembly of C32 with plasmid DNA. After a 10 min incubation period, anionic peptide was added to electrostatically coat the cationic nanoparticles as demonstrated in FIG. 1B. As this self-assembly is driven simply by opposing electrostatic charge, virtually any other cationic polymer (polyethylenimine, poly(β-amino ester), or other) could be potentially used to form similarly coated nanoparticles. Additional details on nanoparticle formation can be found in the materials and methods section in the supporting information. The peptide sequence EEEEEEEEEEEEGGGGGGGRGDS(E12-RGD) (SEQ ID NO: XX) was used as a sequence with specific binding to integrin receptors expressed by HUVECs and the near-identical peptide sequence EEEEEEEEEEEEGGGGGGGRDGS(E12-RDG) (SEQ ID NO: XX) was used as a non-integrin binding control sequence. As the actual fraction of protonated amines in cationic polymeric gene delivery systems is not typically known, charge ratio is frequently reported as a ratio of amine groups on the cationic polymer (N) to phosphate groups on the anionic plasmid DNA (P). Here, we slightly modify the N/P ratio to also include the carboxylic acid groups on the anionic peptide in a manner analogous to what has been done previously.

We use w/w to refer to the weight ratio balance between the polymer and DNA, whereas N/P refers to the overall charge ratio of the nanoparticles, which is variable at a fixed polymer/DNA w/w.

Biophysical Characterization. Previously, we have demonstrated that the size and ξ potential of gene delivery nanoparticles can change depending on the type of aqueous environment in which they are analyzed and that their stability can vary dynamically over time. Furthermore, we have also shown that these changes to biophysical properties directly affect transfection efficacy. Thus in these experiments, the nanoparticles were measured in the actual conditions used during transfection, 12% serum-containing media.

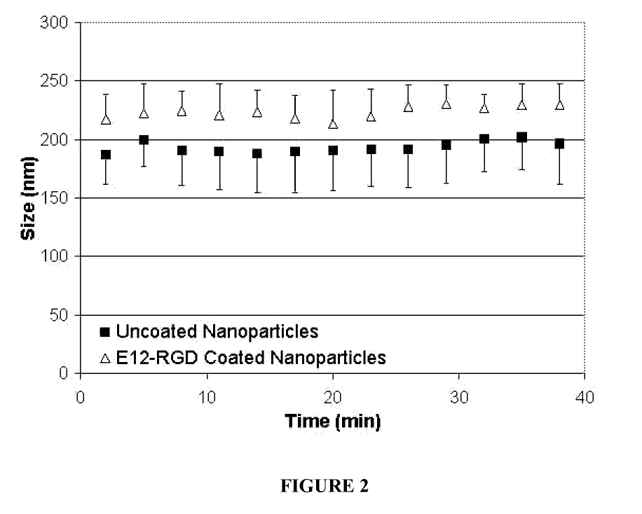

FIG. 2 shows the size and stability of the C32/DNA nanoparticles with and without peptide coating over time. Gene delivery particles are prepared at the same concentrations as they are for typical in vitro bio-assays. This figure demonstrates that particles formed at 50 w/w C32/DNA have a small size of ˜200 nm and are stable in serum over time. It also shows that E12-RGD coating slightly increases particle size while maintaining serum stability. Small size and serum stability are conditions necessary for many therapeutic in vivo applications. Particle stability is also advantageous for consistent transfections and for storage.

FIG. 3 shows the potential of E12-RGD coated and non-coated C32/DNA nanoparticles in 12% serum containing media. The C32/DNA nanoparticles have a negative ξ potential due to serum interactions with the particles. The coated nanoparticles, on the other hand, have a more neutral ξ potential, presumably from the anionic peptide coating reducing serum interactions. Reduction of serum interactions may be beneficial for an in vivo application as serum proteins are known to promote clearance from the blood and to interfere with transfection.

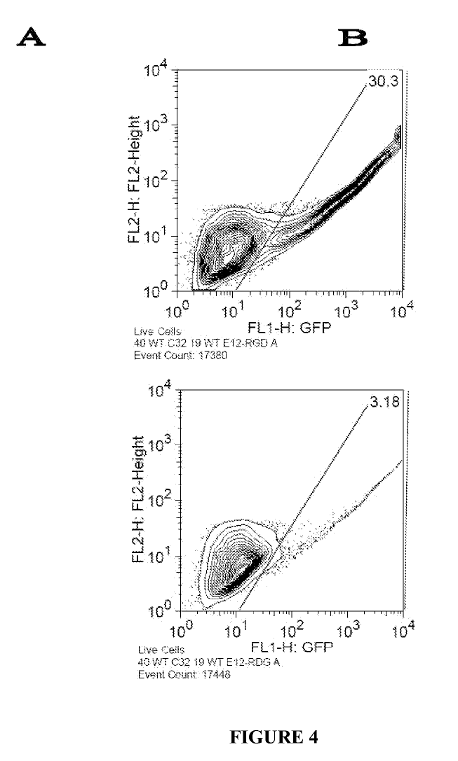

GFP Transfections. Fluorescent Activated Cell Sorting (FACS) and enhanced green fluorescent protein (EGFP) DNA were utilized to determine the efficacy of nanoparticle gene delivery. FACS data was interpreted by using a two-dimensional contour plot that compares the ratio of EGFP channel fluorescence (x-axis) to yellow channel autofluorescence (y-axis) for greater accuracy than a one-dimensional histogram as previously described. FIG. 4 shows the two-dimensional contour plot of representative C32/DNA/E12-RGD and C32/DNA/E12-RDG transfections. These experiments took place under conditions generally seen as difficult for in vitro transfection, but important for an in vivo cardiovascular application: fully confluent, and therefore non-dividing, primary cells in the presence of a high concentration of serum proteins.

Cell Viability. We have previously shown that poly(β-amino ester) nanoparticles are biodegradable and generally non-cytotoxic to multiple cell types including HUVECs. In this study C32/DNA/E12-RGD and C32/DNA/E12-RDG coated nanoparticles were also found to be non-cytotoxic to HUVECs (80%-100% cell viability depending on dose).

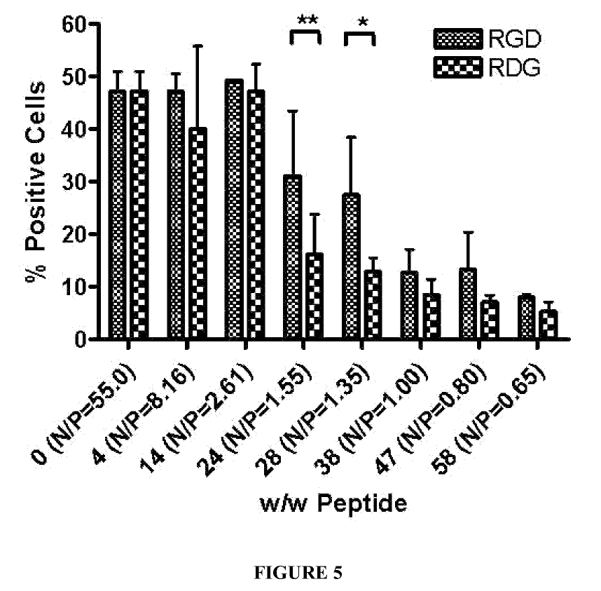

Glutamic Acid-based Coats Enable Ligand-Specific Gene Delivery of Poly(β-amino ester)/DNA nanoparticles to Human Primary Cells. Polyglutamic acid-based peptides were used to coat poly(β-amino ester)/DNA nanoparticles for ligand-based gene delivery. As FIG. 5 shows, at low weights of peptide, the nanoparticles have equivalent transfection whether E12-RGD peptide, E12-RDG peptide, or no coating at all is used. However, at higher weights of anionic peptide, the overall charge ratio of the complexes decreases and efficacy changes occur. When the overall N/P (charge) ratio nears neutrality, E12-RGD coated nanoparticles transfect human endothelial cells significantly better than the same nanoparticles coated with the near identical E12-RDG scrambled sequence, our negative control. Concurrently, as additional anionic peptide coating is added beyond a threshold, overall transfection decreases. Thus, there is a window where (1) the nanoparticles are ligand-specifically targeted and (2) the nanoparticles maintain high efficacy. The results found using this coating system are consistent with previous findings that showed that polylysine conjugated EGF gene delivery particles allow specific binding and internalization only at a relatively narrow window of charge.

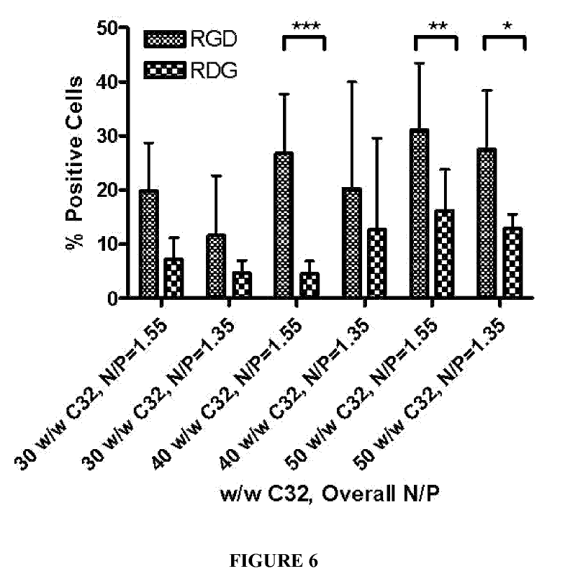

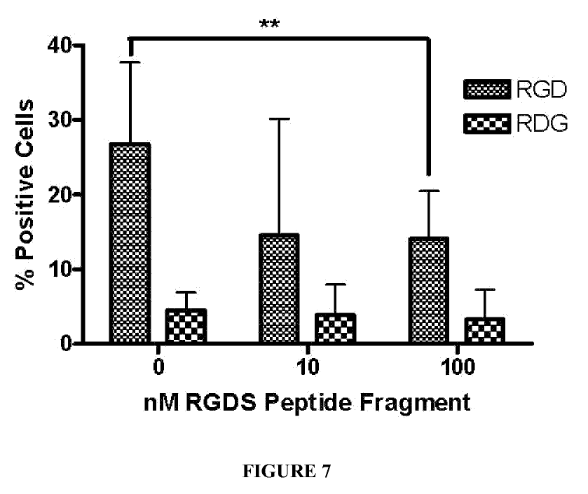

Polymer Weight Ratio, Overall Charge Ratio, and Peptide Length are Important Parameters for Ligand-Specific Gene Delivery. Nanoparticle and coating parameters including polymer weight ratio, peptide weight ratio, overall charge ratio, and peptide length were analyzed to determine optimal conditions. C32/DNA nanoparticle formulations formed at 30 w/w, 40 w/w, and 50 w/w polymer to DNA weight ratios showed the same trends: at low amounts of anionic peptide coating there is no difference in gene delivery between RGD integrin-binding sequence, RDG non-binding sequence, or uncoated controls, but at near neutral N/P ratio, peptide coats formed with RGD integrin-binding sequences delivered DNA much more effectively than the identically formed peptide coated nanoparticles with RDG scrambled sequences. These transfection results of the near-neutrally charged particles can be seen in FIG. 6. Interestingly, at 40 w/w C32/DNA and an N/P ratio of 1.55, integrin-binding E12-RGD coated nanoparticles transfected a six-fold higher percentage of HUVECs as compared to the scrambled sequence E12-RDG coated control particles, our negative control. On an individual cell basis, the most positive cells express GFP at levels 1.000-fold higher than the background. Representative FACS data from these experiments (n>4) can be seen in FIG. 4. The electrostatic ligand coatings provide a mechanism to neutralize nanoparticle charge and reduce non-specific cellular uptake, while simultaneously allowing receptor-specific uptake. To confirm integrin receptor-mediated gene delivery of the RGD coated nanoparticles, fibronectin active fragment peptide RGDS, an integrin binding competitor, was added to the cells prior to transfection. 100 nM RGDS peptide was found to significantly reduce gene delivery of RGD coated nanoparticles (p=0.0043) while not affecting gene delivery of RDG coated nanoparticles (p=0.48) as shown in FIG. 7.

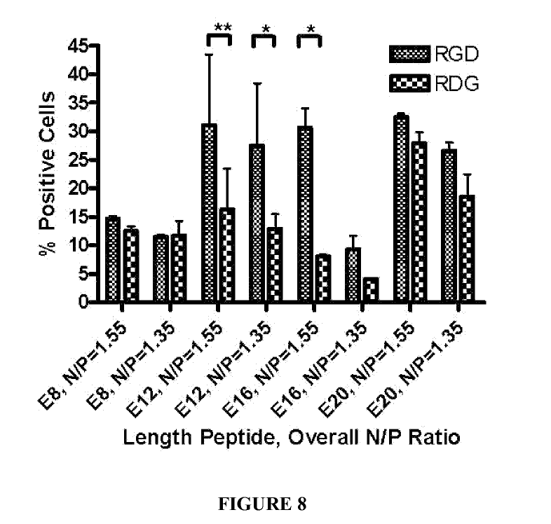

Peptide length was also determined to be important for effective ligand coatings. FIG. 8 demonstrates that while both E12-RGD and E16-RGD coatings allowed for specific delivery, E8-RGD and E20-RGD coatings did not. Thus, only a narrow range of peptide coat lengths (˜12-16 anionic residues plus ligand) is sufficient for ligand-mediated nanoparticle delivery. Interestingly, longer length peptide coating allowed for efficient transfection, even at low N/P ratios, but this transfection was non-specific. Shorter length peptide dramatically reduced transfection efficacy for both RGD and RDG coated nanoparticles suggesting that these short peptide coats neutralize the both the specific and non-specific uptake of these particles.

Discussion. In these experiments, we use Human Umbilical Vein Endothelial Cells (HUVECs) as a model primary cell system due to the following: (1) HUVECs are more difficult to transfect than other cell lines and (2) endothelial cells are prime therapeutic targets against cancer (anti-angiogenesis) and cardiovascular disease (therapeutic angiogenesis, prevention of restenosis, etc). We have also recently shown that poly(β-amino ester) nanoparticles transfect HUVECs significantly better than the leading commercially available non-viral vectors including PEI, jetPEI, and Lipofectamine-2000 and functionalizing these nanoparticles for targeted delivery would further increase their clinical utility. Here we have shown that these nanoparticles can be coated for not only high efficacy, but now also for receptor-mediated delivery.

Previously, electrostatic components have been used with poly(β-amino esters) to construct erodible multilayered films. Here we demonstrate that this concept can be extended to drug delivery nanoparticles to enable single or potentially multi-layered coats for functionalized poly(β-amino ester)-based delivery. Polyacrylic acid and other polycarboxylic acids have also been recently shown to combine with polyethylenimine to form tertiarycomponent gene delivery nanoparticles that have reduced serum inhibition and enhanced nonspecific transfection efficacy. As compared to this technique, our technology enables ligand-specific delivery in a biodegradable system.

This nanoparticle peptide coating approach allows for easy incorporation of a potentially wide range of ligands. Though a peptide sequence containing RGD, such as that used here, can be used for targeting to integrin receptors and/or tumors, many other peptide ligand sequences could be incorporated into this system as well.

We have demonstrated a novel method of coating nanoparticles for ligand-based gene delivery. This method is both flexible and general enough for potentially varied nanoparticle applications. This work also highlights the importance of multiple factors including polymer weight ratio, peptide weight ratio, overall charge ratio, and ligand length when developing coated gene delivery nanoparticles. As this nanoparticulate drug delivery system has high efficacy, ligand-based specificity, biodegradability, low cytotoxicity, and certain safety advantages over viruses, ligand coated poly(β-amino ester) gene delivery nanoparticles may be potentially useful in several clinical applications.

Statistics. Statistical calculations were carried out using GraphPad Prism 4.0 for Windows. Results are reported as mean±standard deviation. For comparison of gene delivery vectors, statistical significance was obtained by using unpaired, two-tailed, Student's t-tests with 95% confidence.

### Materials and Methods

Cell Culture. Human Umbilical Vein Endothelial Cells (HUVECs) (Cambrex, Walkersville, Md., USA) were cultured in EGM-2 media supplemented with SingleQuot Kits (Cambrex). HUVEC cells were used by passage five and in accordance to the manufacturer's instructions. Cells were grown at 37° C. at a humid 5% CO₂ atmosphere.

Polymer Synthesis. Monomers were purchased from Aldrich (Milwaukee, Wis., USA) and Scientific Polymer (Ontario, N.Y., USA). An optimal amine/diacrylate stoichiometric ratio of 1.2:1 for C32 was determined from previous work. To synthesize C32, 400 mg of 5-aminopentanol was weighed into a 1 mL sample vial with a Teflon-lined screw cap. Next, 480 mg of 1,4-butanediol diacrylate was added to the vial along with a small Teflon-coated stir bar. The monomers were then polymerized on a magnetic stir-plate residing in an oven at 95° C. for 1 day. After completion of reaction, the vial was removed from the oven and stored at 4° C. C32 was analyzed by gel-permeation chromatography (GPC) as previously discussed.

Peptide-Coated Polymeric Nanoparticles. Non-viral gene delivery nanoparticles are formed through electrostatic interactions between poly(β-amino ester) C32 and plasmid DNA encoding pEGFP, enhanced green fluorescent protein (ElimBiopharmaceuticals, South San Francisco, Calif., USA). To ease handling, polymer and peptide stock solutions (100 mg/ml) were prepared in DMSO solvent prior to use. Working dilutions of polymer and peptide were prepared in 25 mM sodium acetate buffer (pH 5). To form nanoparticles, 75 μL of diluted polymer was added to an equal volume of DNA, mixed well, and then the mixture was incubated at room temperature for 10 min. 75 μL of anionic peptide EEEEEEEEEEEEGGGGGGGRGDS (SEQ ID NO: XX) or EEEEEEEEEEEEGGGGGGGRDGS (SEQ ID NO: XX) (MIT Biopolymers Laboratory, Cambridge, Mass.) was then mixed with the cationic nanoparticles and the nanoparticles were incubated at room temperature for an additional 5 min. At a fixed basis of DNA and polymer, by varying the weight of peptide coating, the charge ratio was varied.

Biophysical Characterization. Particle size and potential measurements were measured by using a ZetaPALS dynamic light scattering detector (Brookhaven Instruments Corp., Holtsville, N.Y., USA, 15-mW laser, incident beam 676 nm). Correlation functions were collected at a scattering angle of 90°, and particle sizes were calculated using the MAS option of BICTs particle sizing software (version 2.30) using the viscosity and refractive index of water at 25° C. Particle sizes are expressed as effective diameters assuming a log-normal distribution. Average electrophoretic mobilities were measured at 25° C. using BIC PALS potential analysis software, and potentials were calculated using the Smoluchowsky model for aqueous suspensions. Samples were prepared for biophysical characterization in the same manner and at the same concentrations as they were for transfections, but the media/polymer/DNA/peptide solution was scaled up to a final volume of 1.6 mL. Particle stability was determined by changes to particle size over time.

GFP Transfections. Non-viral nanoparticle transfections were performed on confluent HUVECs in the presence of 12% serum. HUVECs were seeded (75,000 cells/well) into clear 24-well plates (Becton Dickinson, Franklin Lakes, N.J., USA) at 24 hours prior to transfection to allow for growth to confluence. Nanoparticles were constructed as previously mentioned using a 6 μg DNA basis per well. Then 150 μL of each polymer/DNA/peptide nanoparticle solution was added to 1.00 mL of FBS supplemented EGM-2 media (12% Serum). The growth medium was removed from the seeded cells using a 6-channel aspirating wand (V&P Scientific, San Diego, Calif., USA) after which 750 μL of the nanoparticle/media solution was immediately added. Nanoparticles were incubated with the cells for 4 hours and then removed using the 6-channel wand and replaced with 500 μL of warm EGM-2 media. After 48 hours, transfected and untransfected control cells were washed, removed from the 24-well plates by trypsinization, microcentrifuged, and resuspended in 500 μL of FACS running buffer (98% PBS/2% FBS/1:200 propidium iodide solution (Invitrogen)) for FACS analysis.

Flow Cytometry. GFP expression was measured using Fluorescence Activated Cell Sorting (FACS) on a FACSCalibur (Becton Dickinson, San Jose, Calif., USA). Propidium iodide staining was used to exclude dead cells from the analysis and 20,000 live cells per sample were acquired. Two-dimensional gating was used to separate increased autofluorescence signal from increased GFP signal to more accurately count positively expressing cells. Gating and analysis was performed using FlowJo 6.3 software (TreeStar, Ashland, Oreg., USA).

Cell Viability Measurements. To measure cytotoxicity and cell viability, cellular metabolic activity was measured using the Cell Titer 96 Aqueous One Solution assay kit (Promega, Madison, Wis., USA). Transfections were performed as described above for 24-well plates, but scaled down five-fold. Metabolic activity was measured using optical absorbance on a Victor3 Multilabel plate counter (Perkin Elmer Life Sciences, Boston, Mass., USA). Measurements of treated cells were converted to percent viability by comparison to untreated controls. HUVECs were treated with a wide range of nanoparticle formulations including a DNA basis of 200 ng-1200 ng per well, 50 w/w C32, and 1.35-55.0 overall N/P ratio depending on level of E12-RGD or E12-RDG coating. Cell viability was determined to be 80%-100% depending on dose.

### Example 2

### Electrostatic Ligand Coatings of Nanoparticles for Nucleic Acid Delivery

E12-PEG-RGD Coatings allow for independent control over the size, charge, and stability of nanoparticles. Covalent PEG attachment to nanoparticles (or to component polymers or biomaterials) have been shown by other researchers to reduce serum interactions and increase circulation time of particles in vivo. Our novel electrostatic approach promises the same benefit of covalent PEG incorporation, but with the ease and generality of self-assembly. This technique also allows for nanoparticles unable to be covalently PEGylated (such as inorganic nanoparticle materials like calcium phosphate) to become PEGylated for multiple uses in drug delivery and other applications. By attaching a ligand at the end of the PEG molecule, specific targeting can be obtained. Blends and layer-by-layer coatings can be used to tune the biophysical properties of the complexes and their overall efficacy.

PEG only (no ligand) coated nanoparticles can reduce particle size in serum vs uncoated nanoparticles by >50%. Optimal coating of 30 w/w C32 is 20 w/w E12-PEG.

Preliminary testing of E12-PEG/E12-PEG-RGD coating blends for targeted gene delivery to HUVECs in serum-containing media.

### Example 3

### Electrostatic Coating Composed of Cationic Peptides/Ligands

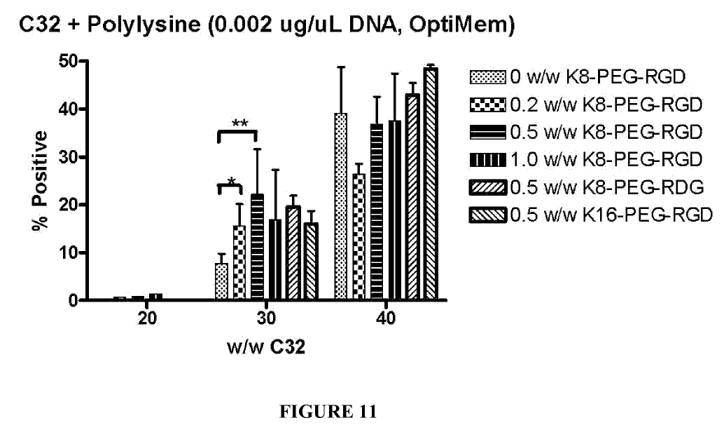

Polylysine-based coats enhance overall transfection efficacy of poly(β-amino ester)/DNA complexes. Cationic, lysine-based peptide coats were found to increase transfection of HUVECs at low weight ratios of polymer to DNA as shown in FIG. 11. At 30 w/w C32/DNA, there is an increase in the percentage of HUVECs transfected in serum by 3-fold depending on the amount of K8-PEG-RGD added. As polymer weight increased, the benefit of adding peptide decreased. At 40 w/w C32/DNA, adding K8-PEG-RGD had no effect on transfection. Changing the length of residues in the peptide coat from K8-PEG-RGD to K16-PEG-RGD produced a similar result at all polymer weight ratios tested. Thus, multiple length ligand coats can enable increased efficacy. Even though the K8-PEG-RGD coating increases transfection, it does not enable ligand specific targeting as the nanoparticles coated with the scrambled K8-PEG-RDG sequence transfected just as well as those nanoparticles coated with the integrin receptor-targeted K8-PEG-RGD sequence. It is hypothesized that this equivalence is due to efficient nonspecific uptake of the positively charged nanoparticles without regard to sequence specific information. Thus at the concentrations used, the PEG spacers were unable to effectively screen the net positive charge of the nanoparticles. To reduce non-specific uptake and allow delivery that is both specific and efficient, a higher percentage of PEG coating and/or a longer length PEG chain as a shield could be used. In addition, incorporation of negatively charged coats (as previously described) could further increase the specificity. The increased transfection efficacy found when adding cationic peptides to low weight ratio polymer/DNA complexes is likely due to enhancement of bottlenecks to gene delivery including DNA binding/protection and/or endosomal escape.

Addition of cationic ligand also allows for control of biophysical properties of the nanoparticles. In serum, weight ratios of 0-1 w/w K8-PEG-RGD reduce overall particle size whereas weight ratios 5-14 w/w increase particle size.

### Example 4

### Use of Drug in the Coating

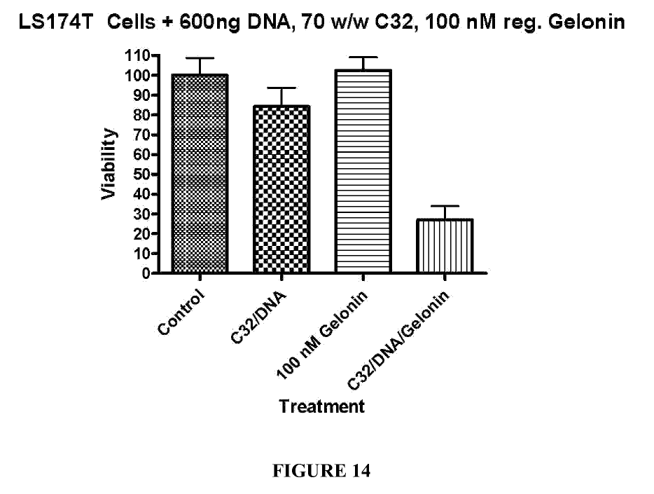

FIG. 14 shows that a single combined treatment of C32/DNA/gelonin killed ˜70% of these colon cancer cells when gelonin on its own was unable to kill any of the cells. Unformulated gelonin is unable to reach the cytoplasm unassisted. In constrast, the gelonin coated nanoparticles are able to be effectively internalized into the cell and released from the endosomal compartment into the cytoplasm, where they are active.

For these experiments, GFP DNA was used to measure gene expression. However, in a future application, DNA containing a therapeutic anti-cancer gene could be used in this system to enable synergistic chemotherapy from the simultaneous gene and drug delivery.

Other peptides, as well as other agents, with or without ligand conjugation, may be coated on C32/DNA nanoparticles for improved drug delivery efficacy and synergistic therapeutic potential.

### Example 5

### Electrostatic Coating Composed of Different Peptide Ligands

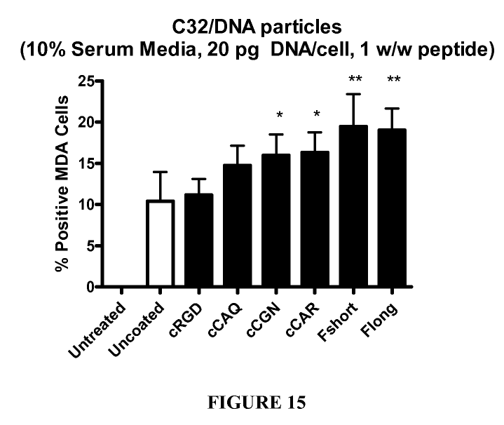

FIG. 15 demonstrates electrostatic coating of particles (C32/DNA) with different peptide ligands. Both linear and cyclic peptides may be used to enhance the delivery of a particle's payload to a target cell. Human MDA breast cancer cells were used in this example to show how electrostatic coating with different peptide ligands can be used to facilitate tumor targeting. DNA-encoding green fluorescent protein (GFP) (20 pg DNA per cell) was delivered to cells, and cells expressing GFP were measured by flow cytometry. The percentages of cells expressing GFP for each ligand as described in the table below are shown in FIG. 15. Uncoated refers to polymeric nanoparticles with any ligand coating. The structure of the coating material was: poly(glutamic acid)-poly(glycine)-X, where X is one of the peptide sequences in the table below.

| Code | Amino Acid Sequence |
|---|---|
| cRGD | CDCRGDCFC |
| | (SEQ ID NO: XX) |
| cCAQ | CAQSNNKDC |
| | (SEQ ID NO: XX) |
| cCGN | CGNKRTRGC |
| | (SEQ ID NO: XX) |
| cCAR | CARSKNKDC |
| | (SEQ ID NO: XX) |
| Fshort | PQRRSARLSA |
| | (SEQ ID NO: XX) |
| Flong | AKVKDEPQRRSARLSAKPAPPKPEPKPKKAPAKK |
| | (SEQ ID NO: XX) |

Statistics: A one-way ANOVA followed by Dunnett's post-test was used to compare each group of ligand-coated particles (black bars) to uncoated control particles (white bar). Data was reported as means plus standard deviation. See FIG. 15.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A coated particle comprising:
a charged particle, wherein the particle comprises a charged polymer and an agent; and
an oppositely charged coating, wherein the coating comprises a charged polymer optionally associated with a targeting agent, a surface modifying agent, or an agent to be delivered; and wherein the coating is adhered to the particle through electrostatic interactions.

2. The coated particle of claim 1 comprising:
a particle, wherein the particle comprises a cationic polymer and an agent; and
a coating, wherein the coating comprises a polyanionic polymer associated with a targeting agent, a surface modifying agent, or a second agent to be delivered; and wherein the coating is adhered to the particle through electrostatic interactions.

3.-7. (canceled)

8. The coated particle of claim 1, wherein the length of the greatest dimension of the particle ranges from approximately 1 nm to approximately 1000 nm.

9. (canceled)

10. The coated particle of claim 2, wherein the cationic polymer comprises a poly(beta-amino ester), a polyamine, a polyethyleneimine, or a cationic protein.

11.-13. (canceled)

14. The coated particle of claim 1, wherein the agent is a polynucleotide, protein, or small molecule.

15.-17. (canceled)

18. The coated particle of claim 2, wherein the polyanionic polymer is a peptide.

19. The coated particle of claim 18, wherein the peptide is polyglutamate, polyaspartate, a peptide comprising both glutamate and aspartate residues, or a peptide consisting of glutamate and aspartate residues.

20.-22. (canceled)

23. The coated particle of claim 2, wherein the particle has a net positive charge.

24. The coated particle of claim 1, wherein the coated particle is approximately neutral in charge.

25. The coated particle of claim 1, wherein the coated particle has a zeta potential ranging from 0 to −10 mV.

26. (canceled)

27. The coated particle of claim 1, wherein the coated particle has a zeta potential ranging from 0 to +10 mV.

28. (canceled)

29. The coated particle of claim 1, wherein the ratio of N/P ranges from approximately 1.2 to approximately 1.6.

30. The coated particle of claim 1, wherein the targeting agent is a peptide, protein, polynucleotide, polysaccharide, small molecule, metal, organometallic complex, antibody, antibody fragment, or aptamer.

31.-36. (canceled)

37. The coated particle of claim 1, wherein the surface modifying agent is a polymer.

38. The coated particle of claim 37, wherein the polymer of the surface modifying agent is polyethylene glycol (PEG) or polyethylene.

39. (canceled)

40. (canceled)

41. The coated particle of claim 1, wherein the second agent is a pharmaceutical agent selected from the group consisting of peptides, proteins, polynucleotides, carbohydrates, and small molecules.

42. (canceled)

43. The coated particle of claim 1, wherein the agent inside the particle is the same as the second agent of the coating.

44. The coated particle of claim 1, wherein the agent inside the particle is different than the second agent of the coating.

45. The coated particle of claim 1 comprising:
a particle, wherein the particle comprises an anionic polymer and an agent; and
a coating, wherein the coating comprises a polycationic polymer associated with a targeting agent, a surface modifying agent, or a second agent to be delivered; and wherein the coating is adhered to the particle through electrostatic interactions.

46. (canceled)

47. (canceled)

48. The coated particle of claim 45, wherein the anionic polymer comprises carboxylic acid, phosphate, or sulfate moieties.

49. The coated particle of claim 45, wherein the polycationic polymer is polyhistidine, polylysine, or polyarginine.

50. (canceled)

51. A pharmaceutical composition comprising a coated particle of claim 1; and a pharmaceutically acceptable excipient.

52. A method of preparing a coated particle, the method comprising steps of:
providing a plurality of particles, wherein the particles have a net charge;
providing an oppositely charged polymer optionally associated with a targeting agent, surface modifying agent, or second agent to be delivered; and
contacting the particles and the polymer under suitable conditions such that the polymer coats the particle through electrostatic interactions.

53.-65. (canceled)

66. A method of administering a coated particle, the method comprising steps of:
providing a coated particle of claim 1; and
administering the coated particle to a subject.

67.-73. (canceled)
